SEQUENCE LISTING (21 sequences)

SEQ ID NO: 1 - DNA, 25 bp, Artificial
  synthetic oligonucleotide
uuuagcaucucuuacgaggacaaug

SEQ ID NO: 2 - DNA, 21 bp, Artificial
  synthetic oligonucleotide
uuacguagggaucugacaggc

SEQ ID NO: 3 - RNA, 25 nt, Artificial
  synthetic oligonucleotide
cauuccuaugcgagacguauguaaa

SEQ ID NO: 4 - RNA, 25 nt, Artificial
  synthetic oligonucleotide
cauuguccucguaagagaugcuaaa

SEQ ID NO: 5 - DNA, 64 bp, Artificial
  synthetic oligonucleotide
cctgttacgtagggatgacaggcgtcagtcagtggccaaaacgcctgtcagatccctacgtaac

SEQ ID NO: 6 - DNA, 64 bp, Artificial
  synthetic oligonucleotide
tgctgttacgtagggatctgacaggcgttttggccactgactgacgcctgtcatccctacgtaa

SEQ ID NO: 7 - protein, 136 aa, Homo sapiens
MARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 8 - protein, 136 aa, Homo sapiens [sequence omitted]

SEQ ID NO: 9 - protein, 136 aa, Homo sapiens
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 10 - protein, 671 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKARDKRDTSNFDKEFTRQPVELTPTDKLFIMNLDQNEFAGFSYTNPEFVINV

SEQ ID NO: 11 - DNA, 2711 bp, Homo sapiens
agctggacgagcggcagcagctgggcgagtgacagccccggctccgcgcgccgcggccgccagagccggcgcaggggaagcgcccgcggccccgggtgcagcagcggccgccgcctcccgcgcctccccggcccgcagcccgcggtcccgcggccccggggccggcacctctcgggctccggctccccgcgcgcaagatggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagctagagacaagagagacacctccaacttcgacaaagagttcaccagacagcctgtggaactgacccccactgataaactcttcatcatgaacttggaccaaaatgaatttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagagggcttttctttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtttttacagttattctataatatcttctttgaatgctaagcatgagcgatatttttaaaaattgtgagtaagctttgcagttactgtgaactattgtctcttggaggaagttttttgtttaagaattgatatgattaaactgaattaatatatgcaa

SEQ ID NO: 12 - protein, 673 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFDRFFTRHPPVLTPPDQEVIRNIDQSEFEGFSFVNSEFLKPEVKS

SEQ ID NO: 13 - DNA, 8014 bp, Homo sapiens
agctggacgagcggcagcagctgggcgagtgacagccccggctccgcgcgccgcggccgccagagccggcgcaggggaagcgcccgcggccccgggtgcagcagcggccgccgcctcccgcgcctccccggcccgcagcccgcggtcccgcggccccggggccggcacctctcgggctccggctccccgcgcgcaagatggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttcgaccgatttttcacccgccatccaccagtcctaacacctcccgaccaggaagtcatcaggaatattgaccaatcagaattcgaaggattttcctttgttaactctgaatttttaaaacccgaagtcaagagctaagtagatgtgtagatctccgtccttcatttctgtcattcaagctcaacggctattgtggtgacatttttatgtttttcattgccaagttgcatccatgtttgattttctgatgagactagagtgacagtgtttcagaacccaaatgtcctcaggtagtttggagcatctctatgagatgggattatgcagatggcctatggaaaatgcagctgcataattaacacattatcaaagtcctcttacaatttattttccgcagcatgtcagctaagtagacccaatggggagagaaaatgcctgctttctttccctctttttctgcactgccatattcacccccaaccatccaatctgtggataattggatgttagcggtactcttccacttccgggcctggagcttggcttgtatccaagtgtatggttgctttgcctaagaggaatccctctatttcacctgttctggaggcaccagaccttgaaaagaacatgctcaaaataaaatgttatctgttatttttgtaaactcaaagttaagatgatcaaagttctaaaattccaagaatgtgcttttagacggtctcaatctaaaagcacttcaaggggtcaaagggcaaccagcttgggtgctacctcagtgttgtagtttctgatactttatgtctttgctcaccctcatccccaaactacttgaaaagggcatttggcaccactctctgaaacaacacagtcactctagcaaggcccccaaagggccctggttttacattacatttcaaactttatttgctttggggttttgtttctgttgttgttcaaatgcaaaaaaaagaaaaaaaaagaaaaaaaaaggtgactcacattgttacacatgctttaaaatatgtattcaaatgttattaaccacaatgacgacctgctttgatttaaccaagaagacggctgcggagcctagcagactcaggcctgtgggaatgggatttgttacaaatctaggtttgttactggcttcagaaagctaattaagtgctctgaaaaagacaccgtttcttgaaacaaagatggttgtattcctcactttgatgttgttttgcaagatgtttgtggaaatgttcatttgtatctggatctctgttatgtgccatttttcttctagcatcgagatacaataaaaaaaaaaaaaaagaaaagaagaagaaatactatttcaaggaaaactgctctttttgagaaacgtggacctaaactacaaagtgggaactgaggagggaactcaggagaaaggaactaactgcggagctttaatcttggccccagtgttcagccactcggaggggcgggggctgtggcccattcaggggctgctggtgggctgtagtggggtgggatgacctggccagagccaacgaggatactggagcccaaagtcaagtttagagaccagctgggaacgtgaatggggctcttgattttcttatcaaaatcaccactcctcccagcttggactaaatattctttctagcaagcagctttgtgagctccctgaagcccaaggaaacccttcggtgggagaaatttcatttctgtctgagaggattaaggcagcaggtgactccccctcctcgcctgccgtgtcctgctattctcaggcagctctaaggagaattcttatcacagttcaagtgatttccagaagttccagggcttctgagagaccatcaagggaactttaacaacttgacaaatgtccttgaagtaagatgcctcatctttagggaaaaatggggtttggatttctgcttaggcaaagtctcctgcagttcatccttctctgtcctcttcttgcttcaggcttggggaccgtccctgctgtccccactgtggtggcaatcaggacctaaggtgaagcaaacttgaagttctatctgacaagtttaggcagtaagagaaggagggaaatcggagcaaagctccctcactttattgttgagaaactggcatctggaaagaggaaggaatttgcccaaagtcagtcagctgggataaaaacctgggtgtcctgtccagaaagtgcagggtgctttctgctctgtagcaaggcagcagacatctctgagccaggcccaccaacaggcccttatctggtggttggatcatgatcccattttgcttggacatgctctcaggaagataaaaaccatggagaaacactaggccattgacaaatgatctgagacaactttagaaaacaatgtaggatgaatggaaagagaaagaaaggaaagaaagaagaaaaagaaagaaggaaagaaagaaagagaaaggaaggaaggaaagaaggaaggaaaagaaggaaggaaggaaggaatatagtgttataaatactgcactcaacattttccaaattcttgccattatttttcaaaagtttaatagtttgcagaaatagatactcaagccaaagtctgttttagagaaactttccatggaaagtcagaatttctaccacttccttttctatccacatttccagtgcagaagaaactgagaaacagagctttttgaagagaggacagggccatagcaacaaggaccttcttgggggattaatgggaggtcagtagaattaataaccctccttggatgagtgctactgttttcacatggcttcagatgctatcaacctcaaagaaatgatctcaacagagaagcttattctctcccaacttctacggtaaaatccaggagtattttctctggggatctgcccacaggacaaagtccataaaagcaagtcctgtctggaccatgtggttatctgaagcattagccatcaccagcacaacaaacggggcagggctttccaaggtggggctggtcagaagggaatctttgataagaggcccacaggcagggaaagcgaaatagggttgatgagaccaggggagacctaaaaaaaaggcagctttgtgtcttctagctccaaatatacctgccttttagctcacacactgtcctggagttctcagacctttaggggccctaacacagttcagttcatacaggggttcaaaagggacagtggcccatttgggagacctttaggatcaatgggaatcaattccattgttttgcctcagagtaaagtttctggctcggggacaattataagttgcaaaaaggatagaggcatatcccaagtcttccttcattccacaaataattacaaacaacctactgtgtgccaggcactattcttagcactggaaatacactagtgaagaagcagatgaggaccctgtttattgtttctctccaagaaattctccaagaatattgtttcttggagagaaataataaataaacaagacaatttctgaaagcaataagtgcaatcaagataattaaaggatgctaaagtgtgacttgtggggattgggagagagatgcacagacaatattaaagaggaggcattcgagctttgttgtgaacaccggaagtaacatgccgagcgcctgggggatggaaactcctatagcaccccacaggctaacagcaagcaggacaagacaaaaagggcaggtgggacatggtagagatggaccctacccaggaaacagctccatcagcatcttagcctgccccactctagccacacatacccacgtgtgctcctgagttcagtgtgcccacctcactcccacaccctcacatagacttggcaagagtaaggagggaactccatagagacattttacctatctcaggggagcagccacaaagaagcaagtcttgtaaaaggtcttttgcaaaggagagtgaacccagcaatgagagatccttaacagctagtgcccattagggggctaaacctaaagcctgggtggtgatggctcaaacgctaatgagtcagtgaatccttaccgaccccctggcctttataatctgaggcaactttggctgcagcccgggaatgtgcagggcactagggaatacaaggccttcttccctggttgtcttgtaataaaacagccatggggttgtccctccagtccgagagactgtgatgaggcctacatagcagcgatgtggtcaggtaaaaatcaggaacccactgaaatcttgggcaagccaccctgcctgcttgtgcctcggttctctcatatgtcatatataggaggtgaggactccagctccacctgccccaggtgggtgtggtgatgatgaggaaagacaagaggcttgcaaggaccctgaagaggtcggagcatcatacagattcctttattagcccacattctgatgttccctggtgagacttgccccaagcaattgctagtaaatgggggttaatttcttctccacctccctactgaacaaaaaaagaaatgccagacttactaggagaatcgagttgctttgagtttcttttgttttgttttgttttgttttgttttaaggctccccttacacaccctcctttaagctttgggttttctctcttatagtttgttgacacatgctaaaaatgtctttggagagaacttctgcctgataaacacccaattctagactgtgggtggattttcgagctgacggtggtcaattcctttcattaagcagtgatctgatttctccacatggccattctgccttcttgggggcagagtagatgggcagcagttcaccttttcagagaaagaggtcttctagccacctgggctgctactgaatggttttctccaggacgctctacctaatgattatttctataacattaagcatggtaataagtagcttccaattcaattcatcctaaagccaaagaaaatacagcaacacacacacacacacacacacacacacacacacacacacacacacaccactttatggcaattcttaactgacattcaatgacttacttcttttcttagaaaatttccaccacatttctatccccaagccaacatacaatgtgaaatgaaagccagtgcgtggagtgcagctgctaaaaattttcagcacagggctctttctgactctgctcatgagatggtatcagccacccaatgactggcgtatcttggtcctgtgtctttcttcttacgctgtgttaatgtgtttactttccatttggcagagagacaagagagacacctccaacttcgacaaagagttcaccagacagcctgtggaactgacccccactgataaactcttcatcatgaacttggaccaaaatgaatttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagagggcttttctttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtttttacagttattctataatatcttctttgaatgctaagcatgagcgatatttttaaaaattgtgagtaagctttgcagttactgtgaactattgtctcttggaggaagttttttgtttaagaattgatatgattaaactgaattaatatatgcaa

SEQ ID NO: 14 - RNA, 25 nt, Artificial
  synthetic oligonucleotide
gaaaguccuagauccacacgcaaau

SEQ ID NO: 15 - RNA, 25 nt, Artificial
  synthetic oligonucleotide
gaacaugauccagaccuacagcaau

SEQ ID NO: 16 - DNA, 25 bp, Artificial
  synthetic oligonucleotide
tttagcatctcttacgaggacaatg

SEQ ID NO: 17 - DNA, 19 bp, Artificial
  synthetic oligonucleotide
gcctgtcatccctacgtaa

SEQ ID NO: 18 - DNA, 21 bp, Artificial
  synthetic oligonucleotide
ttacgtagggatctgacaggc

SEQ ID NO: 19 - DNA, 25 bp, Artificial
  synthetic oligonucleotide
cattgtcctcgtaagagatgctaaa

SEQ ID NO: 20 - protein, 672 aa, Homo sapiens
MADVFPGNDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSVCKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 21 - DNA, 2019 bp, Homo sapiens
atggctgacgttttcccgggcaacgactccacggcgtctcaggacgtggccaaccgcttcgcccgcaaaggggcgctgaggcagaagaacgtgcacgaggtgaaggaccacaaattcatcgcgcgcttcttcaagcagcccaccttctgcagccactgcaccgacttcatctgggggtttgggaaacaaggcttccagtgccaagtttgctgttttgtggtccacaagaggtgccatgaatttgttactttttcttgtccgggtgcggataagggacccgacactgatgaccccaggagcaagcacaagttcaaaatccacacttacggaagccccaccttctgcgatcactgtgggtcactgctctatggacttatccatcaagggatgaaatgtgacacctgcgatatgaacgttcacaagcaatgcgtcatcaatgtccccagcctctgcggaatggatcacactgagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacaaagtcatcagtccctctgaagacaggaaacaaccttccaacaaccttgaccgagtgaaactcacggacttcaatttcctcatggtgttgggaaaggggagttttggaaaggtgatgcttgccgacaggaagggcacagaagaactgtatgcaatcaaaatcctgaagaaggatgtggtgattcaggatgatgacgtggagtgcaccatggtagaaaagcgagtcttggccctgcttgacaaacccccgttcttgacgcagctgcactcctgcttccagacagtggatcggctgtacttcgtcatggaatatgtcaacggtggggacctcatgtaccacattcagcaagtaggaaaatttaaggaaccacaagcagtattctatgcggcagagatttccatcggattgttctttcttcataaaagaggaatcatttatagggatctgaagttagataacgtcatgttggattcagaaggacatatcaaaattgctgactttgggatgtgcaaggaacacatgatggatggagtcacgaccaggaccttctgtgggactccagattatatcgccccagagataatcgcttatcagccgtatggaaaatctgtggactggtgggcctatggcgtcctgttgtatgaaatgcttgccgggcagcctccatttgatggtgaagatgaagacgagctatttcagtctatcatggagcacaacgtttcctatccaaaatccttgtccaaggaggctgtttctgtctgcaaaggactgatgaccaaacacccagccaagcggctgggctgtgggcctgagggggagagggacgtgagagagcatgccttcttccggaggatcgactgggaaaaactggagaacagggagatccagccaccattcaagcccaaagtgtgtggcaaaggagcagagaactttgacaagttcttcacacgaggacagcccgtcttaacaccacctgatcagctggttattgctaacatagaccagtctgattttgaagggttctcgtatgtcaacccccagtttgtgcaccccatcttacagagtgcagtatga

(19) United States
(12) Patent Application Publication
(10) Pub. No.: US 2012/0329854 A1
(43) Pub. Date: Dec. 27, 2012

(54) Methods and Compounds for the Diagnosis and Treatment for Cancer

(75) Inventors: Eric Metzger, Neu-Brisach (FR); Roland Schüle, Weisweil (DE)

(73) Assignee: UNIVERSITÄTSKLINIKUM FREIBURG, Freiburg (DE)

(21) Appl. No.: 13/388,940

(22) PCT Filed: Aug. 3, 2010
(86) PCT No.: PCT/EP10/61317

§ 371 Date: Jul. 26, 2012

(30) Foreign Application Priority Data
Aug. 3, 2009 (EP) 09167060.4

(51) Int. Cl. G01N 33/574; C07H 21/02; C07H 21/04; A61P 35/00; C07K 16/18; A61K 31/713; A61K 31/7105; G01N 33/573; C07K 16/40

(52) U.S. Cl. 514/44 A; 435/7.1; 435/7.4; 436/501; 536/24.5; 536/23.1; 536/24.31; 530/389.7

(57) ABSTRACT

The present invention provides in vitro methods for detecting, grading or prognosticating cancer, in particular prostate cancer. The invention further provides isolated polynucleotides suitable for reducing or inhibiting the expression of protein kinase C beta I and/or II and/or alpha (and consequently the levels of histone H3 phosphorylated at threonine 6, histone H3 monomethylated at lysine 4, histone H3 dimethylated at lysine 4, histone H3 trimethylated at lysine 4) and further relates to pharmaceutical compositions comprising said polynucleotides for the treatment or prevention of cancer, in particular prostate cancer.

### FIELD OF THE INVENTION

The present invention provides in vitro methods for detecting, grading or prognosticating cancer, in particular prostate cancer. The invention further provides isolated polynucleotides suitable for reducing or inhibiting the expression of protein kinase C beta and alpha (and consequently the levels of histone H3 phosphorylated at threonine 6, histone H3 monomethylated at lysine 4, histone H3 dimethylated at lysine 4, histone H3 trimethylated at lysine 4) and further relates to pharmaceutical compositions comprising said polynucleotides for the treatment or prevention of cancer, in particular prostate cancer.

### BACKGROUND OF THE INVENTION

Prostate cancer represents one of the most common types of cancer in men in the developed world and is the second leading cause of male cancer death. Prostate cancer is most often discovered by prostate-specific antigen (PSA) screening and less commonly by physical examination or by symptoms. However, there is some current concern about the accuracy of the PSA test and its usefulness, as said test has been found to often result in false positive or negative results.

Furthermore, the test does not allow the differentiation of benign prostate hyperplasia, non-aggressive prostate cancer and aggressive prostate cancer.

Therefore novel prostate cancer biomarkers are needed in order to improve the early detection and accuracy of diagnosis, to determine the aggressiveness of prostate cancer and to monitor the efficacy of treatment.

The growth and development of prostate cancer is stimulated by androgens (such as e.g. testosterone) and androgen receptor-regulated genes are pivotal to the control of tumour cell proliferation in prostate cancer. Antiandrogens can block the action of androgens and are therefore often employed in hormone therapy during treatment of prostate cancer. Other common treatment options include surgery and radiation. However, all of these treatment methods are associated with a high risk of side effects, including e.g. loss of bladder control, rectal problems, impotence and, in the case of hormone therapy, increased risk of heart attack, weight gain and liver damage. In addition, many patients ultimately become unresponsive to hormonal medications. The development of alternative pharmacological treatments for prostate cancer therefore remains an essential task.

Protein kinase C (PKC) comprises a superfamily of at least 12 isozymes that are activated in response to various stimuli. PKC isozymes are classified as conventional (α, β1, β2, γ), novel (δ, ε, η, θ, μ), and atypical (ζ, λ) isozymes. Conventional PKC isozymes are Ca²⁺-dependent, while novel and atypical isozymes do not require Ca²⁺ for their activation. All PKC isozymes, with the exception of ζ, λ, are activated by diacylglycerol (DAG).

PKC isozymes are serine/threonine kinases that act by catalyzing the transfer of a phosphate group from ATP to serine and threonine residues of their substrate proteins. They phosphorylate a wide variety of intracellular target proteins and have multiple functions in signal transduction-mediated cellular regulation. Activation of one or more PKC iso forms leads to a variety of biological responses, including changes in cell proliferation and differentiation, transmembrane ion transport, glucose and lipid metabolism, smooth muscle contraction, and gene expression. PKC isozymes negatively or positively regulate critical cell cycle transitions, including cell cycle entry and exit and the G₁ and G₂ checkpoints.

Thus, PKCs represent promising targets for the treatment of cancer. However, given the complexity of PKC signaling, general inhibition of all PKC isoforms would result in severe side effects that might even endanger the survival of a patient. Therefore there is an ongoing need for isoform specific therapeutic PKC inhibitors.

### OBJECTIVE AND SUMMARY OF THE INVENTION

It is one object of the present invention to provide methods that can be used for detecting, grading and/or prognosticating cancer, in particular prostate cancer, in a sample from a subject. It is a further object of the present invention to provide pharmaceutical compositions comprising compounds that can be used for the treatment of cancer, in particular prostate cancer.

These and other objectives as they will become apparent from the ensuing description and claims are attained by the subject matter of the independent claims. Some of the preferred embodiments are defined by the dependent claims.

In a first aspect the present invention provides a method for detecting, grading and/or prognosticating cancer comprising the step of determining in a sample from a subject the amount of histone H3 phosphorylated at threonine 6 (H3T6ph), the amount of histone H3 monomethylated at lysine 4 (H3K4me1), the amount of histone H3 dimethylated at lysine 4 (H3K4me2) and/or the amount of histone H3 trimethylated at lysine 4 (H3K4me3).

In a further aspect the present invention provides a method for detecting, grading and/or prognosticating cancer comprising the step of determining in a sample from a subject the expression level of protein kinase C beta 1 and/or 2 and/or alpha (PKCβI and/or II and/or PKCα).

In a preferred embodiment, the present invention relates to a method for detecting, grading and/or prognosticating cancer comprising the step of determining in a sample from a subject the expression level of protein kinase C beta 1 and/or 2 and/or alpha (PKCβI and/or II and/or PKCα); or the expression level of protein kinase C beta 1 and/or 2 and/or alpha (PKCβI and/or II and/or PKCα) and (i) the amount of histone H3 phosphorylated at threonine 6 (H3T6ph); and/or (ii) the amount of histone H3 monomethylated at lysine 4 (H3K4me1); and/or (iii) the amount of histone H3 dimethylated at lysine 4 (H3K4me2); and/or (iv) the amount of histone H3 trimethylated at lysine 4 (H3K4me3).

In another preferred embodiment, the present invention relates to a method for detecting, grading and/or prognosticating cancer comprising the step of determining in a sample from a subject the amount of histone H3 phosphorylated at threonine 6 (H3T6ph); or the amount of histone H3 phosphorylated at threonine 6 (H3T6ph) and (i) the amount of histone H3 monomethylated at lysine 4 (H3K4me1); and/or (ii) the amount of histone H3 dimethylated at lysine 4 (H3K4me2); and/or (iii) the amount of histone H3 trimethylated at lysine 4 (H3K4me3); and/or (iv) the expression level of protein kinase C beta 1 and/or 2 and/or alpha (PKCβI and/or II and/or PKCα).

In another aspect the present invention further provides a diagnostic kit for detecting, grading, and/or prognosticating cancer comprising a detecting agent specific for protein kinase C beta 1 and/or 2 and/or alpha (PKCβI and/or II and/or PKCα), histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3).

In a preferred embodiment, the present invention relates to a diagnostic kit for detecting, grading and/or prognosticating cancer comprising a detecting agent specific for protein kinase C beta 1 and/or 2 and/or alpha (PKCβI and/or II and/or PKCα); or protein kinase C beta 1 and/or 2 and/or alpha (PKCβI and/or II and/or PKCα) and (i) histone H3 phosphorylated at threonine 6 (H3T6ph); and/or (ii) histone H3 monomethylated at lysine 4 (H3K4me1); and/or (iii) histone H3 dimethylated at lysine 4 (H3K4me2); and/or (iv) histone H3 trimethylated at lysine 4 (H3K4me3).

In another preferred embodiment, the present invention relates to a diagnostic kit for detecting, grading and/or prognosticating cancer comprising a detecting agent specific for histone H3 phosphorylated at threonine 6 (H3T6ph); or histone H3 phosphorylated at threonine 6 (H3T6ph) and (i) histone H3 monomethylated at lysine 4 (H3K4me1); and/or (ii) histone H3 dimethylated at lysine 4 (H3K4me2); and/or (iii) histone H3 trimethylated at lysine 4 (H3K4me3); and/or (iv) protein kinase C beta 1 and/or 2 and/or alpha (PKCβI and/or II and/or PKCα).

In yet another aspect the present invention relates to an isolated polynucleotide comprising or consisting of SEQ ID NO: 1 or 2, or a fragment or derivative thereof, wherein said polynucleotide is suitable for inhibiting the expression or activity of protein kinase C beta 1 (PKCβI) and/or protein kinase C beta 2 (PKCβII).

The present invention in a further aspect also relates to a pharmaceutical composition comprising at least one compound selected from
(a) an siRNA molecule suitable for reducing or inhibiting the expression of protein kinase C beta 1 (PKCβI) and/or protein kinase C beta 2 (PKCβII) and/or protein kinase C alpha (PKCα); and/or
(b) the isolated polynucleotide according to the invention.

In yet another aspect the present invention relates to a pharmaceutical composition according to the invention for the treatment or prevention of cancer.

In a further aspect the present invention also relates to the use of a pharmaceutical composition according to the invention for the manufacture of a medicament for the treatment or prevention of cancer.

In yet another aspect the present invention relates to the use of a detecting agent specific for protein kinase C beta 1 and/or 2 and/or alpha (PKCβI and/or II and/or PKCα), histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3) for detecting, grading and/or prognosticating cancer in a sample from a subject.

In a preferred embodiment, the present invention relates to use of a detecting agent specific for protein kinase C beta 1 and/or 2 and/or alpha (PKCβI and/or II and/or PKCα); or protein kinase C beta 1 and/or 2 and/or alpha (PKCβI and/or II and/or PKCα) and (i) histone H3 phosphorylated at threonine 6 (H3T6ph); and/or (ii) histone H3 monomethylated at lysine 4 (H3K4me1); and/or (iii) histone H3 dimethylated at lysine 4 (H3K4me2); and/or (iv) histone H3 trimethylated at lysine 4 (H3K4me3) for detecting, grading and/or prognosticating cancer in a sample from a subject.

In another preferred embodiment, the present invention relates to the use of a detecting agent specific for histone H3 phosphorylated at threonine 6 (H3T6ph); or histone H3 phosphorylated at threonine 6 (H3T6ph) and (i) histone H3 monomethylated at lysine 4 (H3K4me1); and/or (ii) histone H3 dimethylated at lysine 4 (H3K4me2); and/or (iii) histone H3 trimethylated at lysine 4 (H3K4me3); and/or (iv) protein kinase C beta 1 and/or 2 and/or alpha (PKCβI and/or II and/or PKCα) for detecting, grading and/or prognosticating cancer in a sample from a subject.

### BRIEF DESCRIPTION OF THE FIGURES

FIG. 1 PKCα, βI and βII specifically phosphorylate histone H3 at threonine 6 (H3T6). Nucleosomes from HeLa cells (a) or bacterially expressed GST and GST-H3 proteins (b) were incubated with active PKCα, βI, βII, δ, μ, ζ or ι, as indicated. Coomassie blue staining (b, bottom panel) shows the amounts of GST fusion proteins used. Western blots were decorated with the indicated antibodies (a).

FIG. 2 Phosphorylation of H3T6 blocks demethylation at lysine 4 of histone H3 (H3K4) by LSD1. H3K4me2 or H3K4me2T6ph (a) and H3K4me1 or H3K4me1 phosphorylated at threonine 6 (H3K4me1T6ph) (b) peptides corresponding to the H3 tail residues 1-20 were incubated in the presence (a, b; panel 2 and 4) or in the absence of LSD1 (a, b; panel 1 and 3) and analysed by mass spectrometry. A shift in mass equivalent to one methyl group is indicated as “me”.

FIG. 3 PKCβI phosphorylates H3T6 and controls demethylation of H3K4 during AR-dependent gene expression. For ChIP (a-i), LNCaP cells were cultivated in the presence or absence of the AR agonist R1881 and transfected with siRNA (b-i). ChIP analyses were performed with the indicated antibodies. The precipitated chromatin was amplified by PCR using primers flanking androgen response elements (AREs) in the promoter region of the KLK2 and FKBP5 genes or primers in the promoter region of the GAPDH, U6, SCN1A, and SCN2A genes. (j), miRNA-mediated PKCβI knockdown reduces expression of the androgen-regulated KLK2 and FKBP5 genes in LNCaP cells. Bars represent mean±SD (n≧3). Western blots (c, e, j, right panels) were decorated with the indicated antibodies.

FIG. 4 PKCβI, H3T6ph, H3K4me2 and H3K4me3 levels positively correlate with the malignancy of prostate cancer and control androgen-dependent tumour cell proliferation. (a), immunohistochemical staining of AR, PKCβI, H3T6ph, H3K4me2 and H3K4me3 in human normal and tumour prostate. AR (A, B), PKCβI (C, D), H3T6ph (E, F), H3K4me2 (G, H), and H3K4me3 (I, J) immunoreactivity is detected in the secretory epithelium of normal prostate (A, C, E, G, I, arrows) and prostate carcinoma cells (B, D, F, H, J, arrows). All sections were taken from the same radical prostatectomy specimen (magnification: ×250). The correlation of elevated PKCβI, H3T6ph, H3K4me2 and H3K4me3 levels with high Gleason score in a panel of 154 human prostate carcinomas is highly significant: r=0.5292, p<0.0001 (b); r=0.5386, p<0.0001 (c); r=0.5373, p<0.0001 (d); r=0.5395, p<0.0001 (f). Normal prostate specimens are included as a control. (e), miRNA-mediated PKCβI knockdown severely reduces R1881-induced cell proliferation in LNCaP cells. Bars represent mean±SD (n≧6).

FIG. 5 Alphabetical list of the 97 kinases (ProQinase GmbH) used to screen for H3T6 specific kinases.

FIG. 6 The α-H3T3ph²¹, α-H3T6ph (#ab14102 lot 481643; Abcam), α-H3S10ph (#06-570 lot 32219; Upstate), and α-H3T11ph³ antibodies used for Western blot analysis or ChIP assays specifically recognize histone H3 phosphorylated at threonine 3 (H3T3ph), histone H3 phosphorylated at threonine 6 (H3T6ph), histone H3 phosphorylated at serine 10 (H3S10ph), and histone H3 phosphorylated at threonine 11 (H3T11ph), respectively. The indicated amounts of peptides were spotted onto nitrocellulose (Protran BA 79, Schleicher & Schuell). H3T3ph, H3T6ph, H3S10ph, and H3T11ph peptides were obtained from Abcam. The unmodified H3 peptide was obtained from Peptides & Elephants. Western blots were decorated as indicated. Control shows the amounts of Ponceau red stained peptides (bottom panel).

FIG. 7 PKCβI but not PKCα or PKCβII is expressed in LNCaP cells. Western blots were decorated with the indicated antibodies.

FIG. 8 Coomassie blue staining shows the bacterially expressed and purified His-LSD1 protein used in the demethylation assay.

FIG. 9 Coomassie blue staining shows the purified GST and GST-JARID1B protein used in the demethylation assay.

FIG. 10 Phosphorylation of H3T6 blocks demethylation at H3K4 by JARID1B. H3K4me3 or H3K4me3T6ph (a) and H3K4me2 or H3K4me2T6ph (b) peptides corresponding to the H3 tail residues 1-20 were incubated with GST (a, b; panel 1 and 3) or GST-JARID1B (a, b; panel 2 and 4) and analysed by mass spectrometry. A shift in mass equivalent to one methyl group is indicated as “me”.

FIG. 11 The specificity of the α-H3K4me2 and α-H3K4me1 antibodies is not altered by phosphorylation of H3T6. Equal amounts of either untreated nucleosomes (−) or nucleosomes phosphorylated at H3T6 in vitro by PKC (+) were used for Western blot analysis. Western blots were decorated with the indicated antibodies.

FIG. 12 PKCβI is expressed in HeLa cells (a), but does not interact with the chromatinized SCN1A promoter (b). ChIP analyses were performed with the indicated antibodies. The precipitated chromatin was amplified by PCR using primers in the promoter region of SCN1A and GAPDH. Western blots (a) were decorated with the indicated antibodies.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Although several documents are cited throughout the text of this specification, all of which are incorporated herein by reference in their entirety, nothing herein is to be construed as an admission that the invention is not entitled to antedate such disclosure by virtue of prior invention.

The following definitions are introduced:

It is to be understood that the term “comprise”, and variations such as “comprises” and “comprising” is not limiting. For the purpose of the present invention the term “consisting of” is considered to be a preferred embodiment of the term “comprising”. If hereinafter a group is defined to comprise at least a certain number of embodiments, this is meant to also encompass a group which preferably consists of these embodiments only.

The terms “about” and “approximately” in the context of the present invention denote an interval of accuracy that a person skilled in the art will understand to still ensure the technical effect of the feature in question. The term typically encompasses a deviation from the indicated numerical value of ±10% and preferably of ±5%. The determination of percent identity between two sequences is preferably accomplished using the mathematical algorithm of Karlin and Altschul (1993) Proc. Natl. Acad. Sci. USA 90: 5873-5877. Such an algorithm is e.g. incorporated into the BLASTn and BLASTp programs of Altschul et al. (1990) J. Mol. Biol. 215: 403-410 available at NCBI (http://www.ncbi.nlm.nih.gov/blast/Blast.cge). The determination of percent identity is preferably performed with the standard parameters of the BLASTn and BLASTp programs.

BLAST polynucleotide searches are preferably performed with the BLASTn program.

For the general parameters, the “Max Target Sequences” box may be set to 100, the “Short queries” box may be ticked, the “Expect threshold” box may be set to 10 and the “Word Size” box may be set to 28. For the scoring parameters the “Match/mismatch Scores” may be set to 1, −2 and the “Gap Costs” box may be set to linear. For the Filters and Masking parameters, the “Low complexity regions” box may not be ticked, the “Species-specific repeats” box may not be ticked, the “Mask for lookup table only” box may be ticked, the “Mask lower case letters” box may not be ticked.

BLAST protein searches are preferably performed with the BLASTp program.

For the general parameters, the “Max Target Sequences” box may be set to 100, the “Short queries” box may be ticked, the “Expect threshold” box may be set to 10 and the “Word Size” box may be set to “3”. For the scoring parameters the “Matrix” box may be set to “BLOSUM62”, the “Gap Costs” Box may be set to “Existence: 11 Extension: 1”, the “Compositional adjustments” box may be set to “Conditional compositional score matrix adjustment”. For the Filters and Masking parameters the “Low complexity regions” box may not be ticked, the “Mask for lookup table only” box may not be ticked and the “Mask lower case letters” box may not be ticked.

One letter amino acid abbreviations used herein correspond to IUPAC nomenclature (see e.g. European Journal of Biochemistry, 138:9-37, 1984).

The term “RNA interference” or “RNAi” as used herein refers to an RNA induced block of gene expression in a specific and post-transcriptional manner by degradation of a specific target mRNA.

The term “Gleason score” as used herein refers to a means of grading prostate cancer. Cancers with a higher Gleason score are more aggressive and have a worse prognosis. The Gleason score may e.g. be determined based upon the microscopic appearance of a tumor sample. The Gleason score may further be determined by assigning a grade to the most common tumor pattern and a second grade to the next most common tumor pattern in a sample from the tumor and adding the two grades to get the Gleason score. The Gleason grade typically ranges from 1 to 5 with 5 having the worst prognosis, while the Gleason score typically ranges from 2 to 10 with 10 having the worst prognosis.

The terms “cancer” or “tumor” as used herein are meant to include different types of cancers such as e.g. prostate cancer, breast cancer, colorectal cancer, lung cancer, blood cancer and cancers of the brain. Other cancers or tumors may include e.g. non-Hodgkin lymphoma, head and neck cancer, non-small cell lung cancer, ovarian cancer or urinary bladder cancer. One preferred type of cancer in the context of the present invention is prostate cancer. Another preferred type of cancer in the context of the present invention is breast cancer.

The term “isolated” in the context of the present invention indicates that a polynucleotide has been removed from its natural environment and/or is presented in a form in which it is not found in nature.

The term “subject” as used herein preferably refers to a human. However, veterinary applications are also in the scope of the present invention.

The term “subject” can therefore also refer to an animal, preferably a mammal such as e.g. non-human primates, mice, rats, rabbits, guinea pigs, dogs, cats, cattle, horses, sheep, pigs, goats and the like.

The term “aptamer” as used herein refers to a polynucleotide that has a specific binding affinity for a target compound or molecule of interest, e.g. a protein. Aptamers may e.g. be RNA, single stranded DNA, modified RNA or modified DNA molecules. The preparation of aptamers is well known in the art and may involve, inter alia, the use of combinatorial RNA libraries to identify binding sites (reference may e.g. be made to Gold (1995), Ann. Rev. Biochem 64, 763-797).

Histones are the main protein components of chromatin. They form the unit around which DNA is coiled in the nucleosomes of eukaryotic chromosomes. Histone H3 is one of the five main histone proteins involved in the structure of chromatin in eukaryotic cells. Histones may be modified by posttranslational modifications such as e.g. lysine acetylation, lysine methylation, lysine ubiquitylation, arginine methylation or serine, threonine or tyrosine phosphorylation.

The terms “histone H3” or “H3” as used herein may refer to any known histone H3 or variants thereof. Preferably, said histone H3 is mammalian histone H3, most preferably human histone H3.

In some embodiments histone H3 may for example have an amino acid sequence selected from any of SEQ ID NO: 7, 8 or 9 (corresponding to accession number AAA63185.1, accession number NP 066403.2 and accession number CAA88778.1 respectively) or variants thereof. Preferably, said variants are naturally occurring variants. If histone H3 has an amino acid sequence selected from any of SEQ ID NO: 7, 8 or 9, histone H3 may lack the N-terminal methionine residue of said sequence (e.g. due to a post-translational loss of said residue).

Thus, in one preferred embodiment histone H3 has an amino acid sequence selected from any of SEQ ID NO: 7, 8 or 9, wherein said sequence lacks the N-terminal methionine residue.

In some preferred embodiments Histone H3 consists of an amino acid sequence that is at least 70%, 75%, 80%, 85%, 86%, 87%, 88%, 89%, 90%, 91%, 92%, 93%, 94%, 95%, 96%, 97%, 98% or 99% identical to the amino acid sequence according to any of SEQ ID NO: 7, 8 or 9.

The position of amino acid residues within the histone H3 sequence, e.g. threonine 6 (T6) or lysine 4 (K4), is indicated herein with respect to the position of residues in the sequence according to any of SEQ ID NO: 7, 8 or 9, starting from the N-terminal end and sequentially numbering the amino acid residues wherein the number ‘0’ is assigned to the N-terminal methionine residue, which is typically removed from the mature H3 protein in vivo by post-translational modification. In some embodiments where histone H3 has an amino acid sequence different from SEQ ID NO: 7, 8 or 9, the amino acid residues corresponding to threonine 6 and/or lysine 4 (or any other residue of interest) of SEQ ID NO: 7, 8 or 9 may be shifted to another position within said amino acid sequence (e.g. due to amino acid insertions or deletions). In such a case, the amino acids corresponding to threonine 6 and/or lysine 4 (or any other residue of interest) of SEQ ID NO: 7, 8 or 9 may e.g. be determined by aligning the sequence of said histone H3 to any or all of SEQ ID NO: 7, 8 or 9 and detecting the amino acid residues in said sequence which align to threonine 6 and/or lysine 4 (or any other residue of interest) of SEQ ID NO: 7, 8 and/or 9. The skilled person knows how to perform such sequence alignments. For example, blast programs publicly available at NCBI may be used. Preferably, said programs are used with standard parameters.

In one embodiment, threonine 6 and lysine 4 of histone H3 as used in the methods of the invention are comprised in the following consensus sequence of histone H3: ARXKXTAR;

wherein X may be any amino acid.

The inventors of the present invention have inter alia surprisingly found that protein kinase C βI (herein also referred to as PKCβI) phosphorylates histone H3 at threonine 6 and that said phosphorylation blocks demethylation of methylated lysine 4 of histone H3 during androgen receptor (also referred to herein as “AR”) induced gene expression. The inventors of the present invention have inter alia also surprisingly found that elevated levels of protein kinase C βI (PKCβI), histone H3 phosphorylated at threonine 6 (herein also referred to as H3T6ph), histone H3 monomethylated at lysine 4 (herein also referred to as H3K4me1), histone H3 dimethylated at lysine 4 (herein also referred to as H3K4me2) and histone H3 trimethylated at lysine 4 (herein also referred to as H3K4me3) correlate with high Gleason scores in human prostate carcinoma. The present invention thus inter alia provides protein kinase C βI (PKCβI), histone H3 phosphorylated at threonine 6 (H3T6ph), histone H3 monomethylated at lysine 4 (H3K4me1), histone H3 dimethylated at lysine 4 (H3K4me2) and histone H3 trimethylated at lysine 4 (H3K4me3) as novel diagnostic markers for cancer, in particular prostate cancer, which provide the advantage that they can also be used to determine the grade and thus the aggressiveness of a given cancer and to monitor the efficacy of treatment of cancer. Furthermore, several of all of the diagnostic biomarkers according to the invention may be analyzed in parallel within the same sample from a subject thus improving accuracy of detecting cancer in a sample from a subject.

In an especially preferred embodiment, one may thus analyze protein kinase C beta 1 and/or 2 and/or alpha (PKCβI and/or II and/or PKCα) or protein kinase C beta 1 and/or 2 and/or alpha (PKCβI and/or II and/or PKCα) and (i) histone H3 phosphorylated at threonine 6 (H3T6ph); (ii) histone H3 monomethylated at lysine 4 (H3K4me1); and/or (iii) histone H3 dimethylated at lysine 4 (H3K4me2); and/or (iv) histone H3 trimethylated at lysine 4 (H3K4me3); amounts and expression levels, respectively, thereof.

In another especially preferred embodiment, one may thus analyze histone H3 phosphorylated at threonine 6 (H3T6ph) or histone H3 phosphorylated at threonine 6 (H3T6ph) and (i) histone H3 monomethylated at lysine 4 (H3K4me1); and/or (ii) histone H3 dimethylated at lysine 4 (H3K4me2); and/or (iii) histone H3 trimethylated at lysine 4 (H3K4me3); and/or (iv) protein kinase C beta 1 and/or 2 and/or alpha (PKCβI and/or II and/or PKCα); amounts and expression levels, respectively, thereof.

The present invention also provides pharmaceutical compositions comprising an isolated polynucleotide suitable for inhibiting the expression or activity of protein kinase C beta 1 (PKCβI) and/or protein kinase C beta 2 (herein also referred to as PKCβII) for the treatment of cancer.

Said pharmaceutical compositions specifically inhibit protein kinase C β isoforms thus providing the advantage of reduced side effects and of efficient and specific treatment of cancer.

The present invention in a first aspect relates to a method for detecting, grading and/or prognosticating cancer comprising the step of determining in a sample from a subject the amount of histone H3 phosphorylated at threonine 6 (H3T6ph), the amount of histone H3 monomethylated at lysine 4 (H3K4me1), the amount of histone H3 dimethylated at lysine 4 (H3K4me2) and/or the amount of histone H3 trimethylated at lysine 4 (H3K4me3).

The methods according to the invention are preferably performed in vitro.

In one embodiment, determining the amount of H3T6ph, H3K4me1, H3K4me2 and/or H3K4 me3 may e.g. be achieved by contacting the sample from the subject with a detecting agent specific for H3T6ph, H3K4me1, H3K4me2 or H3K4me3 respectively, i.e. the amount of H3T6ph may be detected by contacting the sample from the subject with a detecting agent specific for H3T6ph, the amount of H3K4me1 may be detected by contacting the sample from the subject with a detecting agent specific for H3K4me1, the amount of H3K4me2 may be detected by contacting the sample from the subject with a detecting agent specific for H3K4me2 or H3K4me3 may be detected by contacting the sample from the subject with a detecting agent specific for H3K4me3. In some embodiments the amount of more than one compound selected from the group of H3T6ph, H3K4me1, H3K4me2 and H3K4me3 may be detected in parallel within one sample by applying detecting agents with the respective selectivities. A detecting agent is specific for a given target, if it binds said target with a higher affinity than any other compound in a sample (i.e. a non-target). For example, an antibody is specific for H3T6ph if it binds to H3T6ph with an affinity higher than the affinity with which it binds to H3K4me1, H3K4me2, H3K4me3 or any other compound present in the sample. Preferably, a detecting agent specific for a given target binds to said target only and does not bind at all to a non-target.

A suitable detecting agent may preferably be an antibody or an aptamer. Preferably, the antibody is a monoclonal or polyclonal antibody. In some embodiments the detecting agent may also be selected from antibody variants or fragments such as e.g. single chain antibodies, diabodies, minibodies, single chain Fv fragments (sc(Fv)), sc(Fv)₂ antibodies, Fab fragments or a F(ab′)₂ fragments. In one preferred embodiment commercially available antibodies specific for H3T6ph, H3K4me1, H3K4me2 and/or H3K4me3 may be used. Examples of preferred commercially available antibodies are described herein below in the example section.

Antibodies may be produced according to any suitable method known to the person skilled in the art. Polyclonal antibodies may e.g. be produced by immunization of animals with the antigen of choice, whereas monoclonal antibodies of defined specificity may e.g. be produced using the hybridoma technology developed by Köhler and Milstein (Köhler and Milstein, 1976, Eur. J. Immunol., 6:511-519).

In a preferred embodiment, a detecting agent as described herein above may comprise a detectable label. Any suitable label, which can be attached to the detecting agent may be used. In one preferred embodiment the detectable label is covalently or non-covalently attached to the detecting agent. Examples of labels that may be attached to the detecting agent include e.g. fluorescent dyes such as e.g. Cyanine dyes, e.g. Cyanine 3, Cyanine 5 or Cyanine 7, Alexa Fluor dyes, e.g. Alexa 594, Alexa 488 or Alexa 532, fluorescein family dyes, R-Phycoerythrin, Texas Red and rhodamine. Detecting agents may also be labeled with enzymes such as e.g. horseradish peroxidase, alkaline phosphatase or beta-lactamase, radioisotopes such as e.g. ³H, ¹⁴C, ³²P, ³³P, ³⁵S or ¹²⁵I or metal such as e.g. gold. In another preferred embodiment the detecting agent may also be detected by a secondary detecting agent comprising a label as described above. Preferably a secondary detecting agent is capable of specifically binding to the above described detecting agent. In a particularly preferred embodiment a secondary detecting agent is an antibody.

In some embodiments the amount of H3T6ph, H3K4me1, H3K4me2 and/or H3K4me3 may e.g. be detected in methods involving histological or cell-biological procedures. In some embodiments, visual techniques, such as light microscopy or immunofluoresence microscopy, or flow cytometry or luminometry may be used. In a preferred embodiment H3T6ph, H3K4me1, H3K4me2 and/or H3K4me3 are detected by immunohistochemistry.

In further preferred embodiments determining the amount of H3T6ph, H3K4me1, H3K4me2 and/or H3K4me3 in the sample from the subject may be performed alongside measuring or determining the amount of other compounds or factors, such as e.g. determining the level of prostate-specific antigen (PSA) or Insulin-like Growth Factor-1 (IGF-1) in the same sample or in a different sample from the same subject.

The term “detecting cancer” as used herein means that the presence of a cancerous disease or disorder may be identified in a subject or in a sample from a subject.

Preferably, said subject is previously not known to suffer from cancer. In one preferred embodiment the subject is suspected to suffer from cancer. The term “detecting cancer” as used herein is thus meant to encompass “diagnosing cancer”. The terms “detecting cancer” and “diagnosing cancer” may be used interchangeably. The determination or identification of a cancerous disease or disorder may e.g. be accomplished by comparing the amount of H3T6ph, H3K4me1, H3K4me2 and/or H3K4me3 determined in the sample from the subject to the amount of the respective compound(s) being present in a control as herein described below.

The term “grading cancer” as used herein refers to classifying the cancer by determining certain features of the cancer, such as e.g. its aggressiveness and its prognosis.

The inventors of the present invention have inter alia surprisingly found that elevated levels of protein kinase C β1, H3T6ph, H3K4me1, H3K4me2 and H3K4me3 correlate with high Gleason scores in human prostate cancer. Therefore, in one preferred embodiment “grading cancer”, preferably prostate cancer, in the context of the present invention may be performed by correlating the amount of protein kinase C β1 and/or protein kinase C β2 and/or protein kinase C alpha, H3T6ph, H3K4me1, H3K4me2 and/or H3K4me3 determined in the sample from the subject to cancer grade, preferably prostate cancer grade.

Correlating the amount of protein kinase C β1 and/or protein kinase C α2 and/or protein kinase C alpha, H3T6ph, H3K4me1, H3K4me2 and/or H3K4me3 determined in the sample from the subject to cancer grade may for example be achieved by comparing the amount of protein kinase C β1 and/or protein kinase C β2 and/or protein kinase C alpha, H3T6ph, H3K4me1, H3K4me2 and/or H3K4me3 determined in the sample to previously determined values for the amount of protein kinase C β1 and/or protein kinase C β2 and/or protein kinase C alpha, H3T6ph, H3K4me1, H3K4me2 and/or H3K4me3 respectively, which were determined in one or more control sample(s) from subjects known to suffer from cancer and which were assigned to different tumor grades, preferably to different Gleason scores. The term “prognosticating cancer” as used herein refers to the prediction of the course or outcome of a diagnosed or detected cancerous disease, e.g. during a certain period of time, e.g. during treatment or after treatment. The term may also refer to a determination of chance of survival or recovery from the cancerous disease, as well as to a prediction of the expected survival time of a subject.

For example, the amount of protein kinase C β1 and/or protein kinase C β2 and/or protein kinase C alpha, H3T6ph, H3K4me1, H3K4me2 and/or H3K4me3 may be determined in a sample from a subject at a given point of time and compared to the respective amount of protein kinase C β1 and/or protein kinase C β2 and/or protein kinase C alpha, H3T6ph, H3K4me1, H3K4me2 and/or H3K4me3 determined in a sample from the same subject at a later point of time, wherein an increase or decrease in the amount of protein kinase C β1 and/or protein kinase C β2 and/or protein kinase C alpha, H3T6ph, H3K4me1, H3K4me2 and/or H3K4me3 indicates cancer cell proliferation or cancer regression respectively. Such an approach may e.g. be used during cancer treatment, e.g. during application of anti-cancer medication to a subject suffering from cancer.

Thus in one preferred embodiment the methods according to the invention may be used to monitor the efficacy of cancer treatment in vitro. The efficacy of cancer treatment may e.g. be monitored by detecting the amount of protein kinase C β1 and/or protein kinase C β2 and/or protein kinase C alpha, H3T6ph, H3K4me1, H3K4me2 and/or H3K4me3 in different samples from a subject that were provided over a given period of time while the subject from which the samples were derived was subjected to anti-cancer treatment. An increase or decrease in the amount of protein kinase C β1 and/or protein kinase C β2 and/or protein kinase C alpha, H3T6ph, H3K4me1, H3K4me2 and/or H3K4me3 in samples provided from the subject over a given period of time may then indicate the efficacy of anti-cancer treatment.

In a preferred embodiment the above method further comprises the step of comparing the amount of histone H3 phosphorylated at threonine 6 (H3T6ph), the amount of histone H3 monomethylated at lysine 4 (H3K4me1), the amount of histone H3 dimethylated at lysine 4 (H3K4me2) and/or the amount of histone H3 trimethylated at lysine 4 (H3K4me3) determined in the sample from the subject to the amount of the respective compound(s) determined in a control. The term “respective compound(s)” means that the amount of each compound in the sample is compared to the amount of the respective compound in the control, i.e. e.g. the amount of histone H3 phosphorylated at threonine 6 (H3T6ph) in the sample is compared to the amount of histone H3 phosphorylated at threonine 6 (H3T6ph) in the control, the amount of histone H3 monomethylated at lysine 4 (H3K4me1) in the sample is compared to the amount of histone H3 monomethylated at lysine 4 (H3K4me1) in the control and so on.

In one preferred embodiment the control is a sample from a healthy subject. In a preferred embodiment a higher amount of H3T6ph, H3K4me1, H3K4me2 and/or H3K4me3 determined in the sample from the subject in comparison to the control indicates the presence of cancer in the subject. The term “higher amount” means that the amount of H3T6ph, H3K4me1, H3K4me2 and/or H3K4me3 determined in the sample from the subject is preferably at least 2 fold, at least 3 fold, at least 4 fold, at least 5 fold, at least 10 fold, at least 20 fold, at least 30 fold, at least 40 fold, at least 50 fold, at least 60 fold, at least 70 fold, at least 80 fold, at least 90 fold, at least 100 fold, at least 500 fold, at least 1000 fold or at least 10000 fold higher than in the control.

In some other preferred embodiments, preferably in cases where grading of the cancer is desired, the control may also be a sample derived from a subject known to suffer from cancer, i.e. a subject that has been independently diagnosed with cancer, preferably with prostate cancer or breast cancer. Preferably a control derived from a subject known to suffer from cancer has previously been subjected to cancer grading. Thus, in one preferred embodiment the amount of histone H3 phosphorylated at threonine 6 (H3T6ph), histone H3 monomethylated at lysine 4 (H3K4me1), histone H3 dimethylated at lysine 4 (H3K4me2) and/or histone H3 trimethylated at lysine 4 (H3K4me3) determined in the sample from the subject in comparison to the control may indicate the grade of the cancer present in the subject.

In one preferred embodiment the amount of H3T6ph, H3K4me1, H3K4me2 and/or H3K4me3-in the control may be determined in parallel to the amount of H3T6ph, H3K4me1, H3K4me2 and/or H3K4me3 in the sample from the subject.

In another preferred embodiment the control may be a predetermined value. Such a value may e.g. be based on the results of previous experiments determining the amount of H3T6ph, H3K4me1, H3K4me2 and/or H3K4me3 in one or more samples from a healthy subject or a subject known to suffer from cancer, preferably prostate cancer or breast cancer. In some embodiments a predetermined value may be derivable from a database.

In one preferred embodiment the amount of H3T6ph, H3K4me1, H3K4me2 and/or H3K4me3 may be compared to more than one control, e.g. to 2, 3, 4, 5, 6, 7, 8, 9, 10, 20, 30, 40, 50, 60, 70, 80, 90, 100 or more than 100 controls.

In a further aspect the present invention relates to a method for detecting, grading and/or prognosticating cancer comprising the step of determining in a sample from a subject the expression level of protein kinase C beta 1 and/or 2 and/or alpha (PKCβI and/or II and/or PKCα).

“Determining the expression level of protein kinase C beta 1 and/or 2 and/or alpha” means that the level or amount of protein kinase C beta 1 and/or 2 and/or alpha mRNA and/or protein may be determined. In some embodiments the level of PKC beta and/or alpha pre-mRNA may be determined.

In a preferred embodiment, the term “determining the expression level of protein kinase C beta 1 and/or 2 and/or alpha” means that the enzymatic activity of the protein kinase C beta 1 and/or 2 and/or alpha protein is determined. Clearly, a higher enzymatic activity can result in a higher amount of phosphorylated H3T6, whereas a lower enzymatic activity can be associated with lower amounts of phosphorylated H3T6.

In general, such an assay can include the provision of a sample from the subject (e.g. a tissue sample such as a prostate tissue sample), an optional purification of such a sample, preferably under native conditions in order to retain enzymatic activities of proteins comprised therein, and the analysis of the protein kinase C beta 1 and/or 2 and/or alpha activity from said sample.

It can furthermore be preferred to selectively purify the protein kinase C beta 1 and/or 2 and/or alpha protein from said sample prior to analyzing the kinase activity, e.g. by immunoprecipitations, preferably under native conditions, using corresponding antibodies or the like.

The skilled person is aware of corresponding assays in order to analyze kinase activities. Typically, the substrate (in the present case e.g. histone H3) or a modified and/or truncated and/or synthetic version thereof is provided and the phosphorylation of said substrate or modified and/or truncated and/or synthetic version thereof is quantitatively analyzed using inter alia labeled ATP, such as e.g. fluorescently or radio-labeled ³²P-ATP, antibodies specific for the phosphorylated substrate or the modified and/or truncated and/or synthetic version thereof, or the like.

To this purpose, extracts of tissue samples (preferably freshly prepared; an alternative method would be to immediately freeze such an extract [preferably using liquid nitrogen] at about −20 to −70° C. before performing the activity assay) comprising the protein kinase C beta 1 and/or 2 and/or alpha protein may be incubated with such a substrate or a modified and/or truncated and/or synthetic version thereof. Thus, e.g. an assay as performed in Example 1 of the present application may be carried out.

It is also possible to determine whether the corresponding kinase is in the activated form. As an example in this regard and as shown herein, the activated form of protein kinase C beta 1 is characterized inter alia by phosphorylation at serine 660. Therefore, antibodies specifically recognizing the activated form of protein kinase C beta 1 (i.e. in this example protein kinase C beta 1 phosphorylated at serine 660) or any other detecting agent as set out above detecting the phosphorylated and thus active species may be used in order to detect said activated species. This may e.g. be done in an extract gained as outlined above for the phosphorylation assay.

The amino acid sequences of the protein kinase C beta 1 and/or 2 and/or alpha (PKCβI and/or II and/or PKCα) polypeptides and the nucleotide sequences of the PKC beta and/or alpha pre-mRNA and the protein kinase C beta 1 and/or 2 and/or alpha (PKCβI and/or II and/or PKCα) mRNAs and splice isoforms thereof may be retrieved from any suitable public database such as e.g. the NCBI database (e.g. http://www.ncbi.nlm.nih.gov/pubmed/).

In a preferred embodiment the protein kinase C beta 1 protein has a sequence according to SEQ ID NO: 10 (corresponding to accession number NP_{—}997700.1) and the protein kinase C beta 1 mRNA transcript has a sequence according to SEQ ID NO: 11 (corresponding to accession number NM_{—}212535.2). The protein kinase C beta 1 protein may also comprise or consist of an amino acid sequence being at least 60%, 65%, 70%, 75%, 80%, 85%, 90%, 91%, 92%, 93%, 94%, 95%, 96%, 97%, 98% or 99% identical to the sequence according to SEQ ID NO: 10 and the protein kinase C beta 1 mRNA transcript may comprise or consist of a sequence being at least 60%, 65%, 70%, 75%, 80%, 85%, 90%, 91%, 92%, 93%, 94%, 95%, 96%, 97%, 98% or 99% identical to the sequence according to SEQ ID NO: 11.

In a preferred embodiment the protein kinase C beta 2 protein has a sequence according to SEQ ID NO: 12 (corresponding to accession number NP_{—}002729.2) and the protein kinase C beta 2 mRNA transcript has a sequence according to SEQ ID NO: 13 (corresponding to accession number NM_{—}002738.6).

The protein kinase C beta 2 protein may also comprise or consist of an amino acid sequence being at least 60%, 65%, 70%, 75%, 80%, 85%, 90%, 91%, 92%, 93%, 94%, 95%, 96%, 97%, 98% or 99% identical to the sequence according to SEQ ID NO: 12 and the protein kinase C beta 2 mRNA transcript may comprise or consist of a sequences being at least 60%, 65%, 70%, 75%, 80%, 85%, 90%, 91%, 92%, 93%, 94%, 95%, 96%, 97%, 98% or 99% identical to the sequence according to SEQ ID NO: 13.

In a preferred embodiment the protein kinase C alpha protein has a sequence according to SEQ ID NO: 20 and the DNA coding for protein kinase C alpha has a sequence according to SEQ ID NO: 21. The protein kinase C alpha protein may also comprise or consist of an amino acid sequence being at least 60%, 65%, 70%, 75%, 80%, 85%, 90%, 91%, 92%, 93%, 94%, 95%, 96%, 97%, 98% or 99% identical to the sequence according to SEQ ID NO: 20 and the DNA coding for protein kinase C alpha may comprise or consist of a sequence being at least 60%, 65%, 70%, 75%, 80%, 85%, 90%, 91%, 92%, 93%, 94%, 95%, 96%, 97%, 98% or 99% identical to the sequence according to SEQ ID NO: 21.

The expression level of protein kinase C beta 1 and/or 2 and/or alpha may be determined by any suitable technique known in the art. Determining the amount of mRNA may e.g. be achieved by in situ hybridization, northern blotting, RNAse protection assays and PCR-based methods, such as e.g. reverse transcription PCR and real time quantitative PCR. The skilled person will know how to perform these methods. Determining the amount of protein kinase C beta 1 and/or 2 and/or alpha protein may e.g. be achieved by Western blot analysis, immunohistochemical staining of samples, immunofluorescence and the like (see below for detecting agents which may be used in such assays). Again, the skilled person will know how to perform these methods.

In some embodiments total RNA may be isolated from the sample from the subject prior to determining the amount of mRNA.

In one preferred embodiment the amount of protein kinase C beta 1 and/or 2 and/or alpha mRNA may be detected using a detecting agent specific for protein kinase C beta 1 and/or 2 and/or alpha mRNA. Preferably, said detecting agent is an oligonucleotide probe. In a preferred embodiment, the oligonucleotide probe is a single stranded RNA molecule.

The oligonucleotide probe is specific for protein kinase C beta 1 and/or 2 and/or alpha mRNA if it is capable of hybridizing to protein kinase C beta 1 and/or 2 and/or alpha mRNA under highly stringent conditions.

As used herein the term “hybridize” or “hybridizes” refers to the hybridization of a first to a second polynucleotide. To determine, if two polynucleotides hybridize to each other, the skilled person will preferably conduct hybridization experiments in vitro under moderate or stringent hybridization conditions.

Hybridization assays and conditions are known to those skilled in the art and can be found, for example, in Current Protocols in Molecular Biology, John Wiley & Sons, N.Y., 6.3.1-6.3.6, 1991. Stringent conditions may e.g. be conditions in which hybridization takes place in 6× sodium chloride/sodium citrate (SSC) at 45° C., followed by a wash in 0.2×SSC, 0.1% SDS at 65° C.

Determining the amount of protein kinase beta 1 and/or 2 and/or alpha protein may e.g. be achieved by contacting the sample from the subject with a detecting agent specific for protein kinase beta 1 and/or 2 protein and/or alpha.

A suitable detecting agent may e.g. be an antibody or an aptamer. Preferably, the antibody is a monoclonal or polyclonal antibody. In some embodiments the detecting agent may also be selected from antibody variants or fragments such as e.g. single chain antibodies, diabodies, minibodies, single chain Fv fragments (sc(Fv)), sc(Fv)₂ antibodies, Fab fragments or a F(ab′)₂ fragments. In one preferred embodiment commercially available antibodies against protein kinase C beta 1 and/or 2 and/or alpha may be used. Examples of preferred commercially available antibodies are described herein below in the example section.

In one preferred embodiment the detecting agent specific for protein kinase beta 1 and/or 2 and/or alpha protein may comprise a detectable label. Examples of suitable labels are described herein above.

In another preferred embodiment the detecting agent may also be detected by a secondary detecting agent comprising a label as described above. Preferably a secondary detecting agent is capable of specifically binding to the detecting agent specific for protein kinase beta 1 and/or 2 and/or alpha protein. In a particularly preferred embodiment a secondary detecting agent is an antibody.

In some embodiments the amount protein kinase beta 1 and/or 2 and/or alpha protein may e.g. be detected in methods involving histological or cell-biological procedures.

In some embodiments, visual techniques, such as light microscopy or immunofluoresence microscopy, or cytometry or luminometry may be used. In a preferred embodiment protein kinase beta 1 and/or 2 and/or alpha protein are detected by immunohistochemistry.

In a particularly preferred embodiment the above method for detecting, grading and/or prognosticating cancer comprising the step of determining the expression level of protein kinase C beta 1 and/or 2 and/or alpha (PKCβI and/or II and/or PKCα) in a sample from a subject further comprises the step of comparing the expression level of protein kinase C beta 1 and/or 2 and/or alpha (PKCβI and/or II and/or PKCα) determined in the sample from the subject to the expression level of protein kinase C beta 1 and/or 2 and/or alpha (PKCβI and/or II and/or PKCα) in a control.

In one preferred embodiment the control is a sample from a healthy subject. In a preferred embodiment a higher amount of protein kinase C beta 1 and/or 2 and/or alpha (PKCβI and/or II and/or PKCα) determined in the sample from the subject in comparison to the control indicates the presence of cancer in the subject. The term “higher amount” means that the amount of protein kinase C beta 1 and/or 2 and/or alpha (PKCβI and/or II and/or PKCα) determined in the sample from the subject is preferably at least 2 fold, at least 3 fold, at least 4 fold, at least 5 fold, at least 10 fold, at least 20 fold, at least 30 fold, at least 40 fold, at least 50 fold, at least 60 fold, at least 70 fold, at least 80 fold, at least 90 fold, at least 100 fold, at least 500 fold, at least 1000 fold or at least 10000 fold higher than in the control.

In some other preferred embodiments, preferably in cases where grading of the cancer is desired, the control may also be a sample derived from a subject known to suffer from cancer, i.e. a subject that has been independently diagnosed with cancer, preferably with prostate cancer or breast cancer. Preferably a control derived from a subject known to suffer from cancer has previously been subjected to cancer grading. Thus, in one preferred embodiment the amount of protein kinase C beta 1 and/or 2 and/or alpha (PKCβI and/or II and/or PKCα) determined in the sample from the subject in comparison to the control may indicate the grade of the cancer present in the subject.

In one preferred embodiment the amount of protein kinase C beta 1 and/or 2 and/or alpha (PKCβI and/or II and/or PKCα) in the control may be determined in parallel to the amount of protein kinase C beta 1 and/or 2 and/or alpha (PKCβI and/or II and/or PKCα) in the sample from the subject.

In another preferred embodiment the control may be a predetermined value. Such a value may e.g. be based on the results of previous experiments determining the amount of protein kinase C beta 1 and/or 2 and/or alpha (PKCβI and/or II and/or PKCα) in one or more samples from a healthy subject or a subject known to suffer from cancer, preferably prostate cancer or breast cancer. In some embodiments a predetermined value may be derivable from a database.

In one preferred embodiment the amount of protein kinase C beta 1 and/or 2 and/or alpha (PKCβI and/or II and/or PKCα) may be compared to more than one control, e.g. to 2, 3, 4, 5, 6, 7, 8, 9, 10, 20, 30, 40, 50, 60, 70, 80, 90, 100 or more than 100 controls.

In further preferred embodiments determining the amount of protein kinase beta 1 and/or 2 and/or alpha in the sample from the subject may be performed alongside measuring or determining the amount of other compounds or factors, such as e.g. determining the level of prostate-specific antigen (PSA) or Insulin-like Growth Factor-1 (IGF-1) in the same sample or in a different sample from the subject. In a preferred embodiment determining the amount of protein kinase beta 1 and/or 2 and/or alpha protein in the sample from the subject is performed alongside determining the amount of histone H3 phosphorylated at threonine 6 (H3T6ph), the amount of histone H3 monomethylated at lysine 4 (H3K4me1), the amount of histone H3 dimethylated at lysine-4 (H3K4me2) and/or the amount of histone H3 trimethylated at lysine 4 (H3K4me3) in the same sample or in a further sample from the same subject.

Preferably, the sample from the subject used in the methods according to the invention is separated from the body of the subject. The sample may be solid or liquid. In a preferred embodiment the sample from the subject is a body fluid or tissue sample, preferably a prostate or breast tissue sample. Preferably, the tissue sample is derived from a cancer tissue, most preferably a prostate or breast cancer tissue. Tissue samples may e.g. be fresh or frozen tissue samples or fixed paraffin embedded samples. In one preferred embodiment the sample may be a biopsy or resection sample.

Preferably, the body fluid is blood, plasma, urine, saliva, serum, semen, prostate fluid or seminal fluid.

In some embodiments, a liquid sample may be enriched for cells of interest, e.g. prostate cells. Enrichment may be performed by any method known to the skilled person. Enrichment may e.g. be achieved by using a solid support, e.g. a column, coated with a specific antibody, such as e.g. a prostate specific antibody. Alternatively, enrichment may e.g. also be achieved by using filtration methods or by immobilizing specific aptamers on a microfluidic channel and pumping the liquid sample through the device.

In a further aspect the present invention also relates to a diagnostic kit for detecting, grading, and/or prognosticating cancer comprising a detecting agent specific for protein kinase C beta 1 and/or 2 and/or alpha (PKCβ1 and/or 2 and/or PKCα), histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3).

Preferably, the diagnostic kit according to the invention contains one or more detecting agents specific for protein kinase C beta 1 and/or 2 and/or alpha, histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3). If the diagnostic kit according to the invention comprises more than one detecting agent, said detecting agents are preferably each specific for one compound selected from the group of protein kinase C beta 1 and/or 2 and/or alpha, histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3). Suitable detecting agents have been described herein above. The diagnostic kit may further comprise additional components or reagents that may be suitable for performing the methods according to the invention, such as e.g. buffers or controls. The components contained in the diagnostic kit may be comprised in one or more containers. The diagnostic kit according to the present invention may also comprise an instruction leaflet, which indicates how to use the diagnostic kit and its components.

The present invention in another aspect also relates to an isolated polynucleotide comprising or consisting of SEQ ID NO: 1 or 2 or a fragment or derivative thereof, wherein said polynucleotide is suitable for inhibiting the expression or activity of protein kinase C beta 1 (PKCβI) and/or protein kinase C beta 2 (PKCβII).

SEQ ID NO: 1: uuuagcaucucuuacgaggacaaug
SEQ ID NO: 2: uuacguagggaucugacaggc

As used herein, the term “fragment” generally refers to a polynucleotide of between 10 and 20 nucleotides in length. A fragment may e.g. be 10, 11, 12, 13, 14, 15, 16, 17, 18, 19 or 20 nucleotides in length. A fragment is typically a portion of the polynucleotide it refers to.

As used herein the term “derivative” refers to a polynucleotide sequences that may differ from the polynucleotide sequence it refers to in that one or more nucleotides of the original sequence are substituted by other nucleotides and/or (chemically) modified by methods known to the skilled person, provided that the polynucleotide is still capable of fulfilling its respective function.

A “derivative may comprise between 0 and 5, i.e. 0, 1, 2, 3, 4 or 5 nucleotide substitutions, deletions or insertions, provided that the polynucleotide is still capable of fulfilling its respective function.

The terms “derivative” also includes polynucleotides having linkages between nucleotides that differ from typical linkages. Examples of such polynucleotides specifically include 2′-O-methyl-ribonucleotide, a polynucleotide derivative in which a phosphodiester bond in a polynucleotide is converted to a phosphorothioate bond, a polynucleotides derivative in which a phosphodiester bond in a polynucleotide is converted to a N3′-P5′ phosphoroamidate bond, a polynucleotide derivative in which a ribose and a phosphodiester bond are converted to a pep-tide-nucleic acid bond, a polynucleotide derivative in which uracil is substituted with C-5 propynyl uracil, a polynucleotide derivative in which uracil is substituted with C-5 thiazole uracil, a polynucleotide derivative in which cytosine is substituted with C-5 propynyl-cytosine, a polynucleotide derivative in which cytosine is substituted with phenoxazine-modified cytosine, a polynucleotide derivative in which ribose is substituted with 2′-O-propyl ribose, and a polynucleotide derivative in which ribose is substituted with 2′-methoxyethoxy ribose.

A “derivative” may show at least 50%, preferably at least 60%, preferably at least 70%, preferably at least 75%, at least 76%, at least 77% at least 78% at least 79%, more preferably at least 80%, at least 81%, at least 82%, at least 83%, at least 84%, at least 85%, at least 86%, at least 87%, at least 88% at least 89%, more preferably at least 90%, even more preferably at least 91%, at least 92%, at least 93%, at least 94% least 95%, at least 96%, at least 97% or at least 98% and most preferably at least 99% sequence identity to the polynucleotide sequence it refers to.

The term “reducing or inhibiting” as used herein refers to a reduction in expression of a polypeptide in a cell preferably by more than 30%, more preferably by more than 40%, more preferably by more than 50%, more preferably by more than 55%, more preferably by more than 60%, more preferably by more than 65%, more preferably by more than 70%, more preferably by more than 75%, more preferably by more than 80%, more preferably by more than 85%, more preferably by more than 90%, even more preferably by more than 91%, more than 92%, more than 93%, more than 94%, more than 95%, more than 96%, more than 97%, and most preferably by more than 98% in comparison to the expression of said polypeptide in a control cell. The skilled person knows how to select a suitable control cell. In one embodiment a control cell can e.g. be a cell that has not been treated with the inventive isolated polynucleotide. Comparison is preferably performed under similar experimental conditions.

The isolated polynucleotide according to the invention may be a double stranded or single stranded RNA or DNA molecule. The skilled person will know that in cases where SEQ ID NO: 1 or SEQ ID NO: 2 are DNA, “u” in the sequence is to be replaced by “t”.

In the context of the present invention a DNA molecule comprising SEQ ID NO: 1 or SEQ ID NO: 2 which is used as an insert in an expression vector suitable for the expression of a PKCβ1 or PKCβ2 siRNA or miRNA molecule is also considered a polynucleotide suitable for inhibiting the expression or activity of protein kinase C beta 1 (PKCβI) and/or protein kinase C beta 2 (PKCβII).

Preferably, the isolated polynucleotide according to the invention is a single stranded or double stranded RNA molecule.

In a preferred embodiment, the isolated polynucleotide according to the invention is a small interfering RNA (siRNA), a micro RNA (miRNA), a small hairpin RNA (shRNA) or a dicer-substrate 27-mer duplex.

In a particular preferred embodiment an isolated polynucleotide according to the invention is a small interfering RNA (siRNA) or a micro RNA (miRNA). Said siRNA or miRNA may be single stranded or double stranded.

If the isolated polynucleotide according to the invention is a double stranded RNA, e.g. a double stranded siRNA or a double stranded miRNA, it is preferred that the antisense strand of said double stranded RNA molecule comprises or consists of SEQ ID NO: 1 or 2 or a fragment or derivative thereof and the sense strand of said double stranded RNA molecule comprises or consists of a sequence reverse complement to SEQ ID NO: 1 or 2 or a fragment or derivative thereof.

In one preferred embodiment the isolated polynucleotide according to the invention is suitable for the induction of RNA interference in a cell, preferably a tumor cell. The isolated polynucleotide of the invention preferably has a length of between 10 and 200, more preferably between 12 and 150 nucleotides.

If the polynucleotide according to the invention is siRNA or miRNA, the polynucleotide according to the invention preferably has a length between 12 and 80, between 14 and 60, between 16 and 50, between 17 and 40, more preferably between 18 and 30 nucleotides, most preferably a length of 19, 20, 21, 22, 23, 24, 25, 26, 27, 28, 29 nucleotides.

Expression of an RNA molecule suitable for reducing or inhibiting the expression of protein kinase C beta 1 (PKCβI) and/or protein kinase C beta 2 (PKCβII) may e.g. be achieved by using an expression vector, which includes a polynucleotide sequence that encodes an according siRNA molecule or miRNA molecule. In one preferred embodiment the expression vector expresses an RNA that forms a hairpin with a loop.

The present invention therefore also refers to an isolated polynucleotide, comprising
(a) a first polynucleotide sequence comprising SEQ ID NO: 19 or a fragment or derivative thereof and a second polynucleotide sequence comprising SEQ ID NO: 16 or a fragment or derivative thereof; or
(b) a first polynucleotide sequence comprising SEQ ID NO: 17 or a fragment or derivative thereof and a second polynucleotide sequence comprising SEQ ID NO: 18 or a fragment or derivative thereof.

SEQ ID NO: 16: tttagcatct cttacgagga caatg
SEQ ID NO: 17: gcctgtcatccctacgtaa
SEQ ID NO: 18: ttacgtagggatctgacaggc
SEQ ID NO: 19: cattgtcctcgtaagagatgctaaa

In one preferred embodiment the polynucleotide according to (a) or (b) is double stranded DNA.

In a preferred embodiment the aforementioned isolated polynucleotide according to (a) or (b) is integrated into an expression vector that directs intracellular synthesis of an siRNA molecules or miRNA molecule.

In a preferred embodiment the first polynucleotide sequence is located upstream of the second polynucleotide sequence within the same polynucleotide strand, i.e. SEQ ID NO: 19 or a fragment or derivative thereof is located upstream of SEQ ID NO: 16 or a fragment or derivative thereof or SEQ ID NO: 17 or a fragment or derivative thereof is located upstream of SEQ ID NO: 18 or a fragment or derivative thereof.

In another preferred embodiment a linker polynucleotide is located downstream of the first polynucleotide sequence and upstream of the second polynucleotide sequence, i.e. a linker polynucleotide is located downstream of SEQ ID NO: 19 or a fragment or derivative thereof and upstream of SEQ ID NO: 16 or a fragment or derivative thereof or downstream of SEQ ID NO: 17 or a fragment or derivative thereof and upstream of SEQ ID NO: 18 or a fragment or derivative thereof.

In some other embodiments, the second polynucleotide sequence is located upstream of the first polynucleotide sequence within the same polynucleotide strand, i.e. SEQ ID NO: 16 or a fragment or derivative thereof is located upstream of SEQ ID NO: 19 or a fragment or derivative thereof or SEQ ID NO: 18 or a fragment or derivative thereof is located upstream of SEQ ID NO: 17 or a fragment or derivative thereof.

In such embodiments, if a linker is present, it is preferred that the linker is located downstream of the second polynucleotide sequence and upstream of the first polynucleotide sequence, i.e. a linker is located downstream of SEQ ID NO: 16 or a fragment or derivative thereof and upstream of SEQ ID NO: 19 or a fragment or derivative thereof or downstream of SEQ ID NO: 18 or a fragment or derivative thereof and upstream of SEQ ID NO: 17 or a fragment or derivative thereof.

Preferably the first and/or second polynucleotide sequence has a length of between 10 and 100, between 12 and 80, between 14 and 60, between 16 and 50, between 17 and 40, between 18 and 30 nucleotides, more preferably between 18 and 26 nucleotides. In a particularly preferred embodiment the first and second polynucleotide consist of a sequence according to SEQ ID NO: 19, SEQ ID NO: 16, SEQ ID NO: 17 and SEQ ID NO: 18 respectively.

The terms “upstream” and “downstream” are used herein according to their conventional and well known meaning in the art.

The term “linker polynucleotide” as used herein refers to a polynucleotide sequence that acts as a molecular bridge to operably link two different polynucleotides sequences, wherein one portion of the linker is operably linked to a first polynucleotide sequence, and wherein another portion of the linker is operably linked to a second polynucleotides sequence.

In a preferred embodiment the linker polynucleotide is a DNA sequence. The length of the linker polynucleotide can vary. The linker is preferably 5 to 20 nucleotides in length.

The aforementioned isolated polynucleotide according to (a) or (b) can be single stranded or double stranded. In a preferred embodiment the isolated polynucleotide according to (a) or (b) is double stranded.

In another preferred embodiment an isolated polynucleotide according to the invention may be a morpholino molecule.

Morpholino molecules may for example be used in cases where increased stability of the antisense molecule against cellular nucleases is particularly desirable.

In a preferred embodiment morpholino molecules are used as single-stranded polynucleotides.

In another embodiment, heteroduplexes of a morpholino strand and a complementary polynucleotide strand may be used.

The person skilled in the art will be familiar with the concept of morpholino antisense technology and will know how to synthesize and use suitable morpholino molecules. Reference can e.g. also be made to: Summerton J, Weller D, Antisense Nucleic Acid Drug Dev. 1997 June; 7(3):187-95.

The present invention also relates to an expression vector comprising the aforementioned isolated polynucleotide according to (a) or (b).

In a preferred embodiment the vector allows for the production of double stranded RNA or single stranded RNA. The expression vector may be a prokaryotic or eukaryotic expression vector such as e.g. a plasmid, a minichromosome, a cosmid, a bacterial phage, a retroviral vector or any other vector known to the skilled person. The skilled person will be familiar with how to select an appropriate vector according to the specific need.

One example of a suitable expression vector is pLenti6/V5-DEST (e.g. Invitrogen).

Another example of a suitable expression vector which allows for the production of dsRNA directly in the target cell is the so-called pSUPER (available e.g. from OligoEngine, Inc., Seattle, Wash., United States of America). The vector itself and the mechanism how the dsRNA is produced by using said vector is e.g. described in Brummelkamp et al., 2002, Science, Vol. 296, pages 550-553. Another example of such a vector named pSilencer (available from Ambion) was developed by Sui et al., 2002, Proc. Natl. Acad. Sci. Vol. 99, pages 5515-5520.

The present invention also refers to a host cell comprising an expression vector according to the invention.

Depending on the area of applications, the host cell may be a prokaryotic or eukaryotic host cell. Typical prokaryotic host cells include bacterial cells such as e.g. Escherichia coli (E. coli). Typical eukaryotic host cells include yeast cells such as Saccharomyces cerevisiae, insect cells such as SF9 cells, plant cells and mammalian cells such as COS, CHO and HeLa cells.

The present invention in a further aspect also relates to a pharmaceutical composition comprising at least one compound selected from
(a) an siRNA molecule suitable for reducing or inhibiting the expression of protein kinase C beta 1 (PKCβI) and/or protein kinase C beta 2 (PKCβII) and/or alpha (PKCα); and/or
(b) the isolated polynucleotide according to the invention.

In a preferred embodiment the siRNA molecule suitable for reducing or inhibiting the expression of protein kinase C beta 1 (PKCβI) and/or protein kinase C beta 2 (PKCβII) and/or protein kinase alpha (PKCα) according to (a) is a single stranded or double stranded siRNA molecule that is capable of hybridizing to protein kinase C beta 1 (PKCβI) and/or protein kinase C beta 2 (PKCβII) and/or protein kinase alpha (PKCα) mRNA, thereby inducing RNA interference or any other intracellular antisense mechanism that results in reduction or inhibition of the expression of protein kinase C beta 1 (PKCβI) and/or protein kinase C beta 2 (PKCβII) and/or protein kinase alpha (PKCα).

The siRNA molecule may be of any sequence that allows the siRNA molecule to induce RNA interference resulting in reduction or inhibition of the expression of protein kinase C beta 1 (PKCβI) and/or protein kinase C beta 2 (PKCβII) and/or protein kinase C alpha (PKCα).

Preferably, the siRNA molecule has a length of between 10 and 100, between 12 and 80, between 14 and 60, between 16 and 50, between 17 and 40, more preferably between 18 and 30 nucleotides and most preferably between 18 and 26 nucleotides.

A pharmaceutical composition according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures.

For the production of pills, tablets, sugar-coated tablets and hard gelatin capsules for example, lactose, starch, for example maize starch, or starch derivatives, talc, stearic acid or its salts, etc may be used. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiological sodium chloride solution, alcohols such as ethanol, glycerol, polyols, sucrose, invert sugar, glucose, mannitol, vegetable oils, etc.

In some embodiments the pharmaceutical compositions can also contain additives, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

Examples of suitable excipients for the various different forms of pharmaceutical compositions described herein may e.g. be found in the “Handbook of Pharmaceutical Excipients”, 2nd Edition, (1994), Edited by A Wade and P J Weller.

In some embodiments the pharmaceutical compositions may be sustained release formulations.

In some embodiments the pharmaceutical composition according to the invention in addition to an siRNA molecule suitable for reducing or inhibiting the expression of protein kinase C beta 1 (PKCβI) and/or protein kinase C beta 2 (PKCβII) and/or protein kinase C alpha (PKCα) and/or in addition to the isolated polynucleotide according to the invention may further comprise an active agent suitable for the treatment of cancer. Preferably said active agent is a chemotherapeutic agent.

Examples of suitable chemotherapeutic agents are temozolomide, adriamycin, doxorubicin, epirubicin, 5-fluorouracil, cytosine arabinoside (“Ara-C”), cyclophosphamide, thiotepa, busulfan, cytoxin, taxoids, toxotere, methotrexate, cisplatin, melphalan, navelbine, vinblastine, bleomycin, etoposide, ifosfamide, mitomycin C, mitoxantrone, vincristine, vinorelbine, carboplatin, teniposide, daunomycin, caminomycin, aminopterin, dactinomycin, mitomycins, melphalan andotherrelated nitrogen mustards and hormonal agents that act to regulate or inhibit hormone action on tumors such as tamoxifen and onapristone.

Chemotherapeutic agents which are particularly suited for the treatment of prostate cancer include e.g. leuprolide acetate (Lupron), goserelin acetate (Zoladex), bicalutamide, casodex, cyproterone acetate, flutamide (Eulexin), nilutamide (Nilandron), aminoglutethimide (Cytadren), diethylstilbestrol, finasteride (Proscar), ketoconazole (Nizoral), megestrol acetate (Megace), suramin, estramustine phosphate sodium (Emcyt), taxanes, prednisone, paclitaxel, doxorubicin, zoledronic acid, pamidronate, atrasentan, estramustine phosphate aminogluthetimide, ketoconazole, corticosteroids, etoposide, cyclophosphamide, degarelix, (Novantrone), docetaxel (Taxotere), paclitaxel (Taxol).

The pharmaceutical composition according to the invention may also additionally comprise a further inhibitor of protein kinase C beta 1 and/or 2 activity, such as e.g. LY 317615, CGP41251, Gö66983, LY333531, Ro 317549, Bisindolylmaleimide I or Ro318220.

In other preferred embodiments separate compositions comprising the aforementioned active agents may be administered to a subject in combination with a pharmaceutical composition comprising an siRNA molecule suitable for reducing or inhibiting the expression of protein kinase C beta 1 (PKCβI) and/or protein kinase C beta 2 (PKCβII) and/or protein kinase C alpha (PKCα) and/or the isolated polynucleotide according to the invention.

In a further aspect the present invention also relates to a pharmaceutical composition according to the invention for the treatment or prevention of cancer.

In a preferred embodiment said cancer is prostate cancer.

The present invention also relates to the use of a pharmaceutical composition according to the invention for the manufacture of a medicament for the treatment or prevention of cancer.

In a preferred embodiment said cancer is prostate cancer.

The present invention in a further aspect also relates to the use of a detecting agent specific for protein kinase C beta 1 and/or 2 and/or alpha (PKCβI and/or II and/or PKCα), histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3) for detecting, grading and/or prognosticating cancer in a sample from a subject. Preferably, said use in vitro.

“Protein kinase C beta 1 and/or 2 and/or alpha (PKCβI and/or II and/or PKCα)” in this context is meant to refer to protein kinase C beta 1 and/or 2 and/or alpha protein and/or mRNA.

Suitable detecting agents are described herein above. Regarding detecting agents for detecting protein kinase C beta 1 and/or 2 and/or alpha (PKCβI and/or II and/or PKCα), it can be particularly preferred to use a detecting agent capable of detecting whether the kinases are in the activated form. Thus, one may e.g. use an antibody specific for detecting the S660-phosphorylated species of protein kinase C 1 in order to detect not only the kinase but also the activation state of said protein.

In another aspect the present invention relates to the diagnostic kit according to the invention or the use of a detecting agent specific for protein kinase C beta 1 and/or 2 and/or alpha (PKCβI and/or II and/or PKCα), histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3) for detecting, grading and/or prognosticating cancer in a sample from a subject, wherein the detecting agent is an antibody, an aptamer or an oligonucleotide probe.

Examples of suitable antibodies, aptamers and oligonucleotide probes have been described herein above.

In a further aspect the present invention relates to the methods according to the invention, the diagnostic kit according to the invention, the pharmaceutical composition according to the invention and/or the use according to the invention, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer. In a particularly preferred embodiment the cancer is prostate cancer.

Further preferred embodiments of the present invention relate to:
1. A method for detecting, grading and/or prognosticating cancer comprising the step of determining in a sample from a subject the amount of histone H3 phosphorylated at threonine 6 (H3T6ph), the amount of histone H3 monomethylated at lysine 4 (H3K4me1), the amount of histone H3 dimethylated at lysine-4 (H3K4me2) and/or the amount of histone H3 trimethylated at lysine 4 (H3K4me3).
2. A method according to 1, further comprising the step of comparing the amount of histone H3 phosphorylated at threonine 6 (H3T6ph), the amount of histone H3 monomethylated at lysine 4 (H3K4me1), the amount of histone H3 dimethylated at lysine 4 (H3K4me2) and/or the amount of histone H3 trimethylated at lysine 4 (H3K4me3) determined in the sample from the subject to the amount of the respective compound(s) determined in a control.
3. A method for detecting, grading and/or prognosticating cancer comprising the step of determining in a sample from a subject the expression level of protein kinase C beta 1 and/or 2 (PKCβI and/or II).
4. A method according to 3, further comprising the step of comparing the expression level of protein kinase C beta 1 and/or 2 (PKCβI and/or II) determined in the sample from the subject to the expression level of protein kinase C beta 1 and/or 2 (PKCβI and/or II) in a control.
5. The method according to any of 1 to 4, wherein the sample from the subject is a body fluid or tissue sample, preferably a prostate or breast tissue sample.
6. The method according to 5, wherein the body fluid is selected from the group of blood, plasma, urine, saliva, serum, semen, prostate fluid or seminal fluid.
7. A diagnostic kit for detecting, grading and/or prognosticating cancer comprising a detecting agent specific for protein kinase C beta 1 and/or 2 (PKCβI and/or II), histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3).
8. An isolated polynucleotide comprising or consisting of SEQ ID NO: 1 or 2 or a fragment or derivative thereof, wherein said polynucleotide is suitable for inhibiting the expression or activity of protein kinase C beta 1 (PKCβI) and/or protein kinase C beta 2 (PKCβII).
9. An isolated polynucleotide according to 8, wherein said isolated polynucleotide is a small interfering RNA (siRNA) or a mico RNA (miRNA).
10. A pharmaceutical composition comprising at least one compound selected from
   (a) an siRNA molecule suitable for reducing or inhibiting the expression of protein kinase C beta 1 (PKCβI) and/or protein kinase C beta 2 (PKCβII); and/or
   (b) the isolated polynucleotide according to any of 8 or 9.
11. A pharmaceutical composition according to 10 for the treatment or prevention of cancer.
12. Use of a pharmaceutical composition according to 10 for the manufacture of a medicament for the treatment or prevention of cancer.
13. Use of a detecting agent specific for protein kinase C beta 1 and/or 2 (PKCβI and/or II), histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3) for detecting, grading and/or prognosticating cancer in a sample from a subject.
14. The diagnostic kit according to 7 or the use according to 13, wherein the detecting agent is an antibody, an aptamer or an oligonucleotide probe.
15. The method according to any of 1 to 6, the diagnostic kit according to 7, the pharmaceutical composition according to 11 and/or the use according to any of 12 to 13, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.

Still further preferred embodiments of the present invention relate to:
1. A method for detecting, grading and/or prognosticating cancer comprising the step of determining in a sample from a subject the amount of histone H3 phosphorylated at threonine 6 (H3T6ph); or the amount of histone H3 phosphorylated at threonine 6 (H3T6ph) and (i) the amount of histone H3 monomethylated at lysine 4 (H3K4me1); and/or (ii) the amount of histone H3 dimethylated at lysine 4 (H3K4me2); and/or (iii) the amount of histone H3 trimethylated at lysine 4 (H3K4me3); and/or (iv) the expression level of protein kinase C beta 1 and/or 2 and/or alpha (PKCβI and/or II and/or PKCα).
2. The method according to 1, further comprising the step of comparing the amount of histone H3 phosphorylated at threonine 6 (H3T6ph); or the amount of histone H3 phosphorylated at threonine 6 (H3T6ph) and (i) the amount of histone H3 monomethylated at lysine 4 (H3K4me1); and/or (ii) the amount of histone H3 dimethylated at lysine 4 (H3K4me2); and/or (iii) the amount of histone H3 trimethylated at lysine 4 (H3K4me3); and/or (iv) the expression level of protein kinase C beta 1 and/or 2 and/or alpha (PKCβI and/or II and/or PKCα) determined in the sample from the subject to the amount of the respective compound(s) determined in a control.
3. The method according to 1 or 2, wherein the sample from the subject is a body fluid or tissue sample, preferably a prostate or breast tissue sample.
4. The method according to 3, wherein the body fluid is selected from the group of blood, plasma, urine, saliva, serum, semen, prostate fluid or seminal fluid.
5. The method according to any of 1 to 4, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.
6. A diagnostic kit for detecting, grading and/or prognosticating cancer comprising a detecting agent specific for histone H3 phosphorylated at threonine 6 (H3T6ph); or histone H3 phosphorylated at threonine 6 (H3T6ph) and (i) histone H3 monomethylated at lysine 4 (H3K4me1); and/or (ii) histone H3 dimethylated at lysine 4 (H3K4me2); and/or (iii) histone H3 trimethylated at lysine 4 (H3K4me3); and/or (iv) protein kinase C beta 1 and/or 2 and/or alpha (PKCβI and/or II and/or PKCα).
7. The diagnostic kit according to 6, wherein the detecting agent is an antibody, an aptamer or an oligonucleotide probe.
8. The diagnostic kit according to 6 or 7, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.
9. Use of a detecting agent specific for histone H3 phosphorylated at threonine 6 (H3T6ph); or histone H3 phosphorylated at threonine 6 (H3T6ph) and (i) histone H3 monomethylated at lysine 4 (H3K4me1); and/or (ii) histone H3 dimethylated at lysine 4 (H3K4me2); and/or (iii) histone H3 trimethylated at lysine 4 (H3K4me3); and/or (iv) protein kinase C beta 1 and/or 2 and/or alpha (PKCβI and/or II and/or PKCα) for detecting, grading and/or prognosticating cancer in a sample from a subject.
10. The use according to 9, wherein the detecting agent is an antibody, an aptamer or an oligonucleotide probe.
11. The use according to 9 or 10, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.

Other preferred embodiments of the present invention relate to:
1. A method for detecting, grading and/or prognosticating cancer comprising the step of determining in a sample from a subject the amount of histone H3 phosphorylated at threonine 6 (H3T6ph); or the amount of histone H3 phosphorylated at threonine 6 (H3T6ph) and (i) the amount of histone H3 monomethylated at lysine 4 (H3K4me1); and/or (ii) the amount of histone H3 dimethylated at lysine 4 (H3K4me2); and/or (iii) the amount of histone H3 trimethylated at lysine 4 (H3K4me3); and/or (iv) the expression level of protein kinase C beta 1 and/or 2 (PKCβI and/or II).
2. The method according to 1, further comprising the step of comparing the amount of histone H3 phosphorylated at threonine 6 (H3T6ph); or the amount of histone H3 phosphorylated at threonine 6 (H3T6ph) and (i) the amount of histone H3 monomethylated at lysine 4 (H3K4me1); and/or (ii) the amount of histone H3 dimethylated at lysine 4 (H3K4me2); and/or (iii) the amount of histone H3 trimethylated at lysine 4 (H3K4me3); and/or (iv) the expression level of protein kinase C beta 1 and/or 2 (PKCβI and/or II) determined in the sample from the subject to the amount of the respective compound(s) determined in a control.
3. The method according to 1 or 2, wherein the sample from the subject is a body fluid or tissue sample, preferably a prostate or breast tissue sample.
4. The method according to 3, wherein the body fluid is selected from the group of blood, plasma, urine, saliva, serum, semen, prostate fluid or seminal fluid.
5. The method according to any of 1 to 4, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.
6. A diagnostic kit for detecting, grading and/or prognosticating cancer comprising a detecting agent specific for histone H3 phosphorylated at threonine 6 (H3T6ph); or histone H3 phosphorylated at threonine 6 (H3T6ph) and (i) histone H3 monomethylated at lysine 4 (H3K4me1); and/or (ii) histone H3 dimethylated at lysine 4 (H3K4me2); and/or (iii) histone H3 trimethylated at lysine 4 (H3K4me3); and/or (iv) protein kinase C beta 1 and/or 2 (PKCβI and/or II).
7. The diagnostic kit according to 6, wherein the detecting agent is an antibody, an aptamer or an oligonucleotide probe.
8. The diagnostic kit according to 6 or 7, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.
9. Use of a detecting agent specific for histone H3 phosphorylated at threonine 6 (H3T6ph); or histone H3 phosphorylated at threonine 6 (H3T6ph) and (i) histone H3 monomethylated at lysine 4 (H3K4me1); and/or (ii) histone H3 dimethylated at lysine 4 (H3K4me2); and/or (iii) histone H3 trimethylated at lysine 4 (H3K4me3); and/or (iv) protein kinase C beta 1 and/or 2 (PKCβI and/or II) for detecting, grading and/or prognosticating cancer in a sample from a subject.
10. The use according to 9, wherein the detecting agent is an antibody, an aptamer or an oligonucleotide probe.
11. The use according to 9 or 10, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.

The present invention will now be described with respect to some of its specific examples. These examples are however not to be construed in a limiting way.

### EXAMPLES

### Material and Methods

### Plasmids

The following plasmids were described previously: pGEX-4T1-H3 1-15, pGEX-4T1-H3 16-30, pGEX-4T1-H3 29-44, pET-GEX H3 1-135, and pLenti6-miRNA-control³. GST-H3 1-135 T6A was provided by J.M.G. Higgins. To construct GST H3 1-15 T6A the corresponding cDNA fragment was cloned into pGEX-4T1. To construct pLenti6-miRNA-PKCβI, the DNA corresponding to miRNA-PKCβI (5′-TGCTGTTACGTAGGGATCTGACAGGCGTTTTGGCCACTGACTGACGCCTGTCATCCCTACGTAA-3′ (SEQ ID NO:6) and 5′-CCTGTTACGTAGGGATGACAGGCGTCAGTCAGTGGCCAAAACGCCTGTCAGATCCCTACGTAAC-3′) (SEQ ID NO:5) was cloned into pLenti6N5-DEST according to the manufacturer's instructions (Invitrogen).

### Chromatin Immunoprecipitation

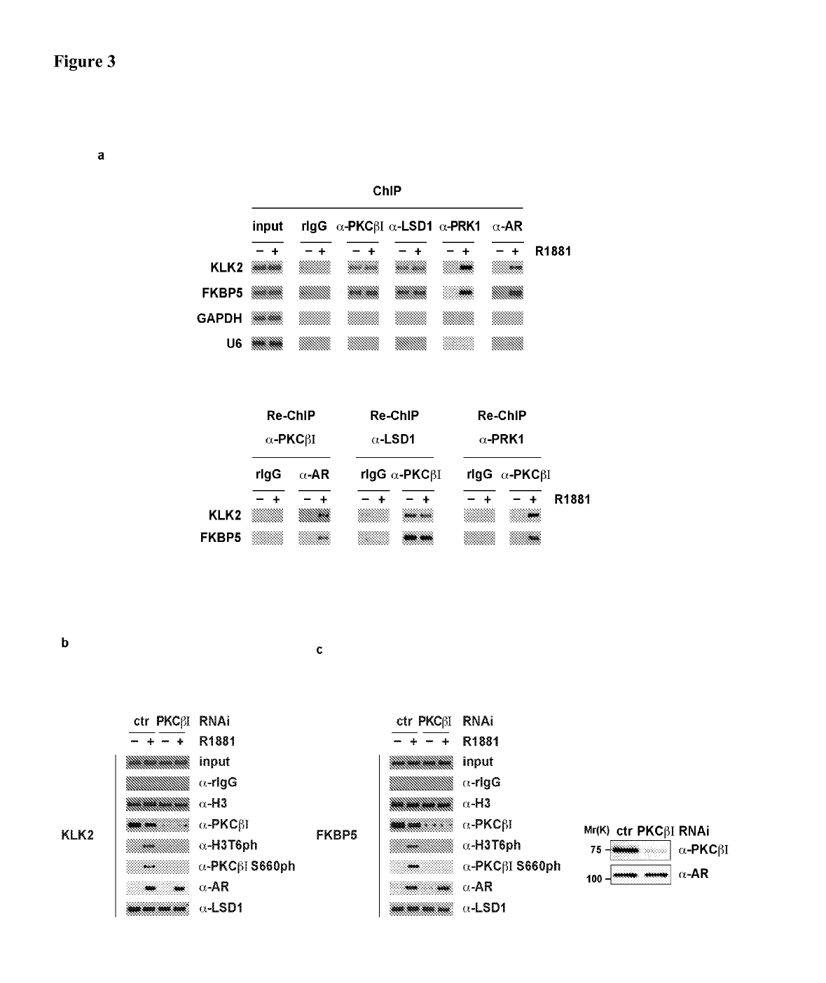

ChIP and Re-ChIP experiments were performed as described³. LNCaP cells were cultured for 15 min (ChIP) or 210 min (Re-ChIP) in the presence or absence of 1×10⁻⁸M R1881 as indicated. Three days before harvesting, cells were transfected with siRNA (control for PKCβI: 5′-CAUUCCUAUGCGAGACGUAUGUAAA-3′(SEQ ID NO 3); PKCβI: 5′-CAUUGUCCUCGUAAGAGAUGCUAAA-3′ (SEQ ID NO 4) (FIG. 3 b, c, f-i); control for PRK1: 5′-GAAAGUCCUAGAUCCACACGCAAAU-3′ (SEQ ID NO 14); PRK1: 5′-GAACAUGAUCCAGACCUACAGCAAU-3′ (SEQ ID NO: 15) (FIG. 3 d, e); Invitrogen) using RNAifect (Qiagen) following the manufacturer's instructions. Immunoprecipitation was performed with specific antibodies (α-AR #06-680 lot 33529 (Upstate Biotechnology), α-H3K4me1 #ab8895 lot 535654, α-H3T6ph #ab14102 lot 481643, α-H3 #ab1791 lot 172452, α-PKCβI S660ph #ab23513 lot 596904 (Abcam), α-PKCβI (C-16) #sc-209 lot 481643, (Santa Cruz), α-H3K4me2 #cs-035-100 lot A391001 (Diagenode), α-PRK1³, and α-LSD1²) on protein A-Sepharose 4B (GE-Healthcare). For PCR, 2 μl out of 50 μl DNA extract was used. PCR primers for FKBP5 (+351/+443), KLK2 (−343/−90), SCN1A, SCN2A, GAPDH, and U6 were described previously^{1, 2, 6, 11}.

### Western Blot Analysis

Experiments were performed as described². Western blots were decorated as indicated.

### Cell Proliferation Assay

Experiments were performed as described². pLenti6-miRNA-control and pLenti6-miRNA-PKCβI were used to produce recombinant lentiviruses to infect LNCaP cells as described¹⁵. The infected cells were cultured for 72 hours in medium containing 10% double-stripped FCS. 1×10⁴ cells were plated in a 96-well plate in the presence or absence of 1×10⁻⁹ M R1881. The cell proliferation Elisa BrdU Colorimetric Assay (Roche) was performed according to the manufacturer's instructions. The figure shows the increase of proliferation in the presence versus absence of R1881. The experiments were performed in hexaplicate.

### Quantitative RT-PCR and Statistical Analysis

Quantitative RT-PCR and statistical analysis were performed as described. The primers for KLK2 and FKBP5 were described previously^{6, 11}.

### In Vitro Kinase Assay

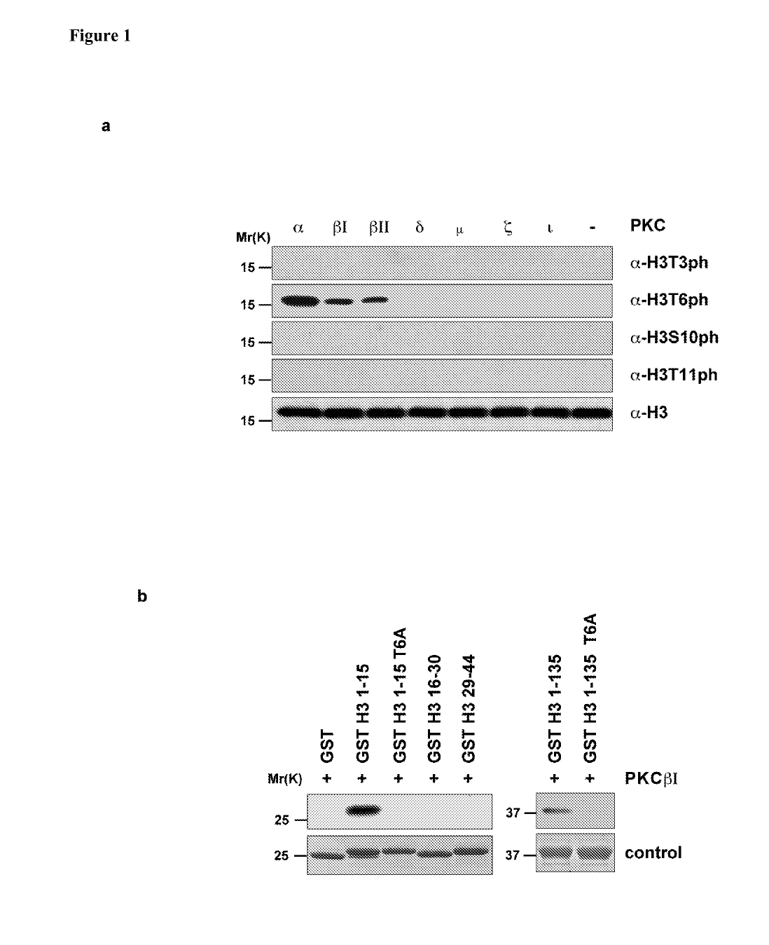

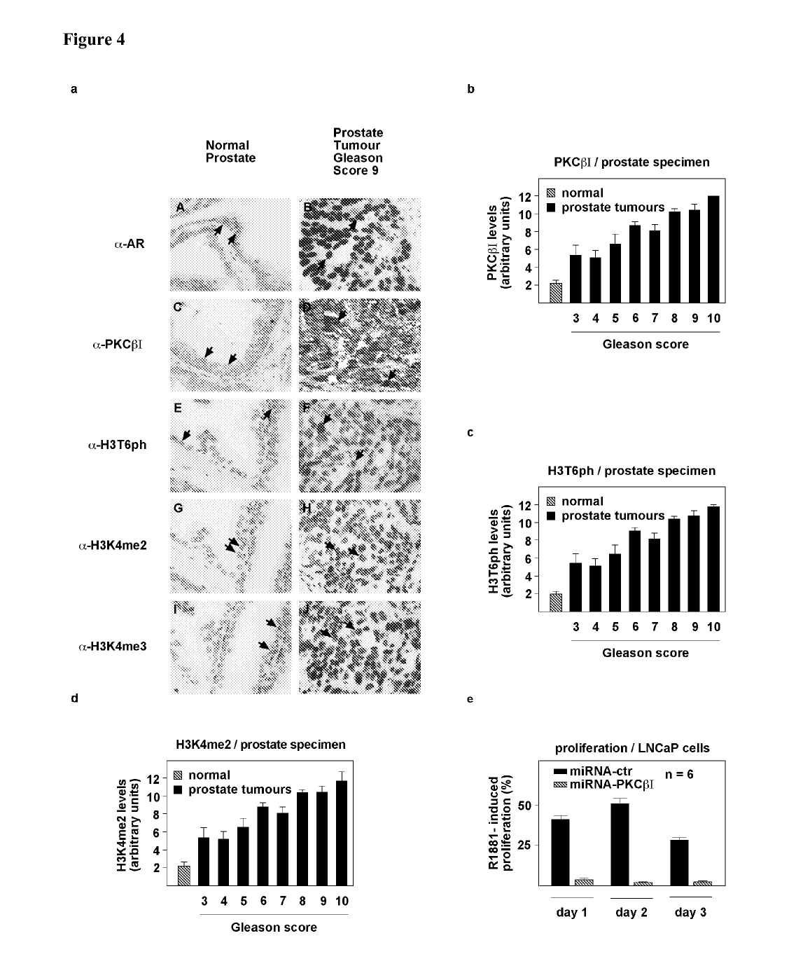

The kinase assays were performed as described³. Ten μg of either GST-H3 mutants (FIG. 1b), or 1 μg of nucleosomes (FIG. 1a) purified from HeLa cells¹⁶ were incubated with the amount corresponding to 12 pmol/min⁻¹ purified recombinant PKC (ProQinase) at 30° C. in kinase buffer containing 60 mM Hepes-NaOH pH 7.5, 3 mM MgCl₂, 3 mM MnCl₂ and either 10 mM ATP (FIG. 1a) or 5 μCi [γ-³²P]ATP (FIG. 1b). The reaction mixture was analysed by Western blotting using antibodies as indicated (FIG. 1a) or SDS-PAGE followed by autoradiography (FIG. 1b).

### Immunohistochemistry

Stainings were performed using a protocol¹⁷ for antigen retrieval and indirect immunoperoxidase. α-AR (#sc-7305 lot E171; Santa Cruz), α-PKCβI (#sc-209 lot 481643; Santa Cruz), α-H3T6ph (#ab14102 lot 481643; Abcam), and α-H3K4me2 (#ab7766-100, lot 56290; Abcam) antibodies were used at a dilution of 1:75, 1:1000, 1:200, and 1:1000, respectively. Immunoreactions were performed with the universal vectastain ABC kit according to the manufacturer's instructions (Vector Laboratories).

### Tissue Microarrays and Statistical Analysis

Clinical data of patients and procedures for generating the tissue microarrays were described previously¹⁷. Tissue microarrays were prepared from formalin-fixed, paraffin-embedded tissue specimens of 154 prostates selected from the archival files of the Institute of Pathology, University of Bonn Medical School. All tumour samples were surgically obtained from patients who had undergone radical retropubic prostatectomy in two surgical centres between 1995 and 2002 for clinically organ confined prostate cancer (preoperative staging </=cT2, cNo, cMo). Patients who had received prior hormonal therapy, chemotherapy, or radiation therapy were excluded from the study. All cases were re-evaluated by a panel of experienced pathologists for histopathological staging according to the UICC TNM-system¹⁸, rescored according to the Gleason scoring system¹⁹ and subsequently followed-up between 21 to 128 months (median 40.24 months). Three different tissue cores representing the lowest and highest Gleason grades within a single tumour were arrayed from formalin-fixed, paraffin-embedded tissue blocks using a manual device (Beecher Instruments). 4 μm paraffin sections were cut from every TMA and used for subsequent immunohistochemical analyses within one week. Statistical analysis was performed with the Mann-Whitney U-Test using the SPSS 12.0 program (SPSS Inc.) and by calculating the two-tailed Spearman Rank correlation coefficient. The number of cases (n) analysed per Gleason score (Gs) were: Gs 3 (n=5); Gs 4 (n=12); Gs 5 (n=14); Gs 6 (n=38); Gs 7 (n=24); Gs 8 (n=27); Gs 9 (n=20); Gs 10 (n=14). Normal prostate specimens (n=25).

### Demethylase Assay and Mass Spectrometry Analysis

The demethylation assays were performed essentially as described^{1, 20}. One μg peptides corresponding to the H3 tail residues 1-20 carrying either one, two, or three methyl groups at K4 in the absence or presence of a phosphorylated T6 were incubated with 5 μg bacterially expressed and purified His-LSD1, or 5 μg baculovirus expressed and purified GST-JARID1B and GST as indicated. Peptides were incubated for 5 hours at 37° C. in demethylation buffer containing 50 mM Tris HCl pH 7.5, 50 mM KCl, 5 mM MgCl₂ or 50 mM HEPES-KOH pH 8.0, 2 mM ascorbate, 100 μM Fe(NH₄)₂(SO₄)₂, 1 mM α-ketoglutarate^{1, 20}. The crude demethylase reactions were diluted 1:10 in a saturated CHCA solution (50% acetonitril, 0.1% TFA) and spotted onto a MALDI-target plate. For each reaction 2000 spectra were recorded and analysed using the Data Explorer Software™

### Example 1

### H3T6 is Phosphorylated by PKCβI

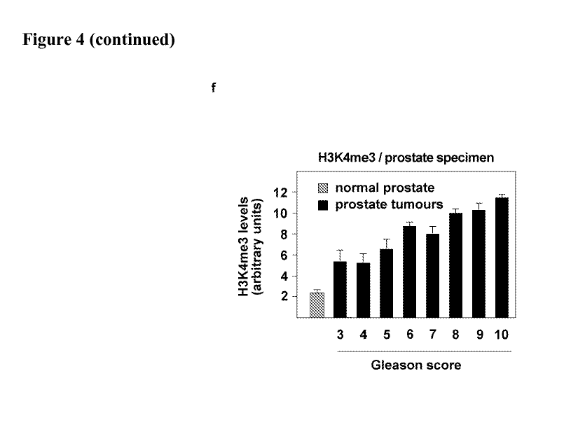

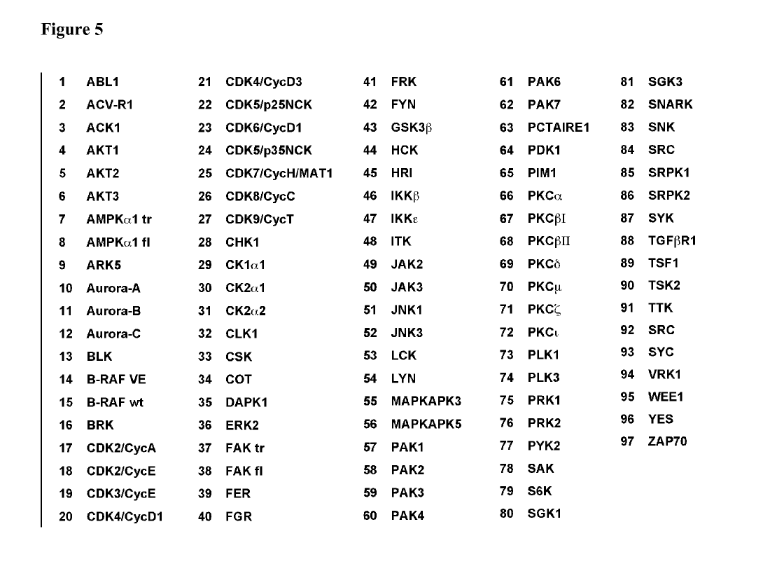

To identify putative H3T6 kinases, in vitro kinase screens were performed by incubating nucleosomes purified from HeLa cells with 97 recombinantly expressed, purified kinases (FIG. 5). Western blot analysis performed with antibody specific to phosphorylated H3T6 (α-H3T6ph) demonstrates that only PKCα, 131 and 132 phosphorylate nucleosomes at H3T6 (FIG. 1a). PKCα,β1 and β2 specifically target H3T6 but not threonine 3, serine 10, or threonine 11 of histone H3 (H3T3, H3S10, H3T11, respectively) (FIG. 1a). Specificity of the antibodies was verified (FIG. 6). Western blot analysis performed with α-PKCα, β1 or β2 antibodies shows that in LNCaP human prostate cancer cells only PKCβI is expressed (FIG. 7). Therefore, all further studies were continued with PKCβI. To corroborate in an independent approach that PKCβI exclusively phosphorylates H3T6, PKCβI was incubated with recombinant, purified GST-H3 or GST control proteins in the presence of [γ-³²P]ATP. In these experiments only full-length histone H3 (H3 1-135) and a H3 fragment spanning amino acid residues 1 to 15 (H3 1-15), but not H3 16-30 or H3 29-44, are phosphorylated by PKCβI (FIG. 1b). Furthermore, mutation of threonine 6 to alanine in either H3 1-15 (H3 1-15 T6A) or in full length H3 (H3 1-135 T6A) abolishes phosphorylation, demonstrating that PKCβI targets H3T6 only (FIG. 1b). Taken together, said data identify PKCβI as the first H3T6 kinase.

### Example 2

### Phosphorylation of H3T6 Blocks the Demethylation of H3K4 Methyl Marks

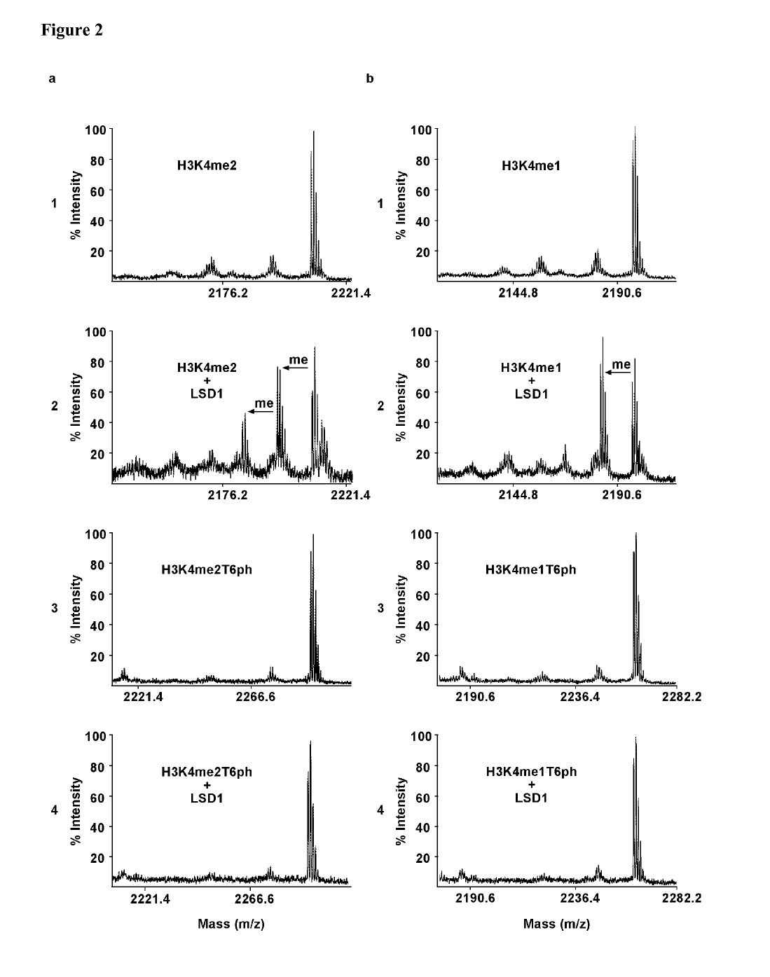

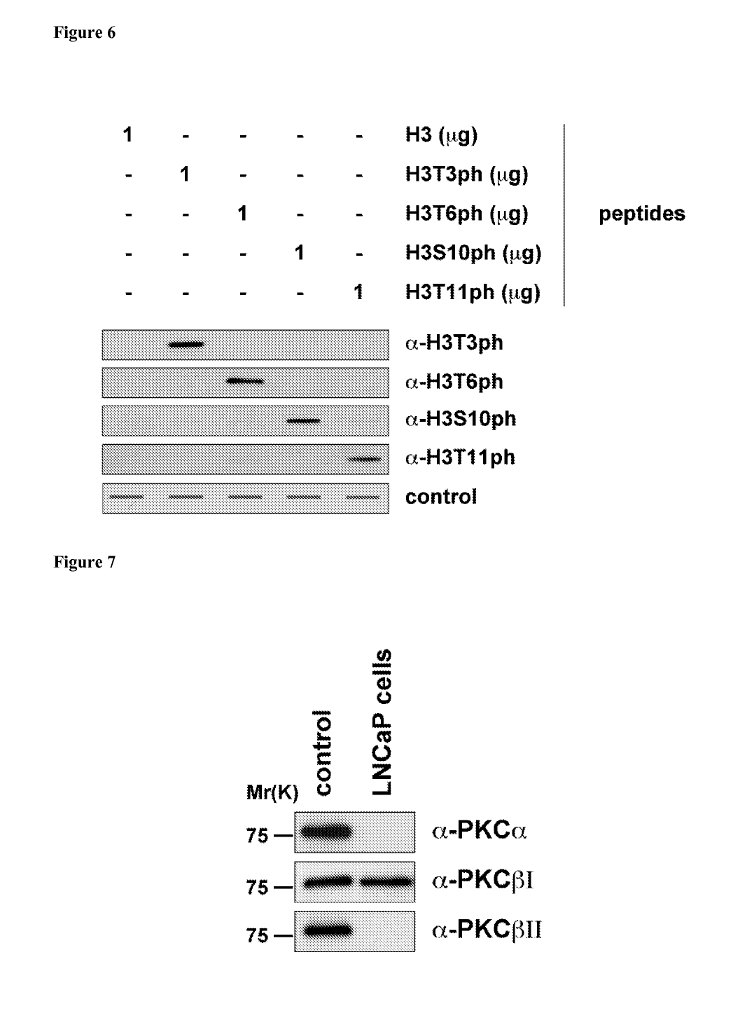

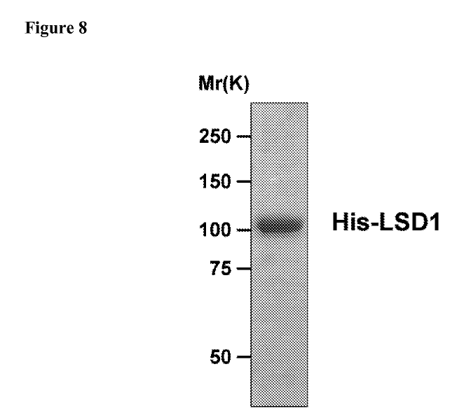

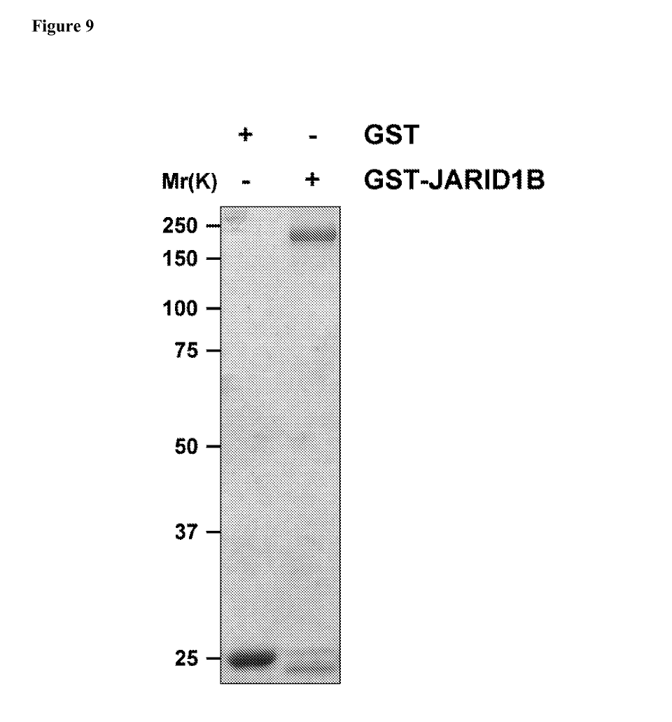

Demethylation assays were performed with di-, and monomethyl H3K4 peptides either unmodified at T6 (H3K4me2 and H3K4me1) or phosphorylated at T6 (H3K4me2T6ph and H3K4me1T6ph) and recombinant, purified LSD1 (FIG. 2 a, b and FIG. 8). Following the demethylation assay, the peptides were analysed by mass spectrometry. The robust demethylation of H3K4me2 observed in the presence of LSD1 (FIG. 2 a, compare panels 1 and 2) is completely blocked when the peptides are phosphorylated at T6 (FIG. 2 a, compare panels 3 and 4). Likewise, demethylation of H3K4me1 by LSD1 is completely abrogated in the presence of phosphorylated T6 (FIG. 2 b, panels 3 and 4). Since LSD1 only removes mono- and dimethyl marks^{1, 2}, it was tested whether phosphorylation at H3T6 also blocks demethylation of tri-, and dimethyl H3K4 (H3K4me3 and H3K4me2) by members of the KDM5 family of JMJC domain-containing demethylases such as JARID1B/KDM5B⁹. Recombinant, purified JARID1B (FIG. 9) efficiently demethylates H3K4me3 and H3K4me2 peptides (FIG. 10 a, b, compare panels 1 and 2). In contrast, demethylation of tri- and dimethyl H3K4 peptides phosphorylated at T6 (H3K4me3T6ph and H3K4me2T6ph) is severely impaired (FIG. 10 a, b, compare panels 3 and 4). These results demonstrate that phosphorylation of H3T6 blocks demethylation of H3K4 methyl marks in vitro.

### Example 3

### PKCβI and LSD1 form a Complex on Chromatin in a Ligand-Dependent Manner

Western blot analysis performed with anti-PKC α, β1 or β2 antibodies indicates that only PKCβI is expressed in LNCaP cells (FIG. 7). Therefore, it was investigated whether PKCβI is present at AR target genes and phosphorylates H3T6 in vivo. To analyse first whether PKCβI binds to promoters of AR target genes, LNCaP cells were subjected to chromatin immunoprecipitation (ChIP) in the presence or absence of the AR agonist R1881. PKCβI specifically associates with the androgen response elements (AREs) located in the promoters of the KLK2¹⁰ and FKBP5¹¹ genes (FIG. 3a, upper panel). Association of PKCβI with AR target promoters is specific since DNA from neither GAPDH nor U6 promoters is enriched (FIG. 3a, upper panel). PKCβI binds to chromatin in a ligand-independent manner similar to LSD1 (ref² and see FIG. 3a, upper panel). In comparison, Protein-kinase-C-related kinase 1 (PRK1) and AR bind chromatin only in a ligand-dependent manner. To investigate whether PKCβI and AR are present in the same complex on the KLK2 and FKBP5 promoters, R1881-treated LNCaP cells were subjected to sequential chromatin immunoprecipitation (Re-ChIP), first with α-AR and then with α-PKCβI antibody. PKCβI and AR form a complex on chromatin upon addition of ligand (FIG. 3a, lower panel). Similarly, it was investigated whether PKCβI and LSD1 are present in the same complex on the KLK2 and FKBP5 promoters. Re-ChIP first with α-PKCβI and then with α-LSD1 antibody demonstrates that PKCβI and LSD1 form a complex on chromatin in a ligand-independent manner (FIG. 3a, lower panel).

### Example 4

### H3T6ph is a Novel Chromatin Mark for Transcriptional Activation

Next, it was investigated whether PKCβI phosphorylates H3T6 at promoters of AR-regulated genes in vivo. LNCaP cells, cultured in the presence or absence of R1881, were transfected with either an unrelated control siRNA or a siRNA directed against PKCβI, and then subjected to ChIP with α-H3T6ph antibody. Importantly, addition of ligand results in phosphorylation of H3T6 at the AREs of the KLK2 and FKBP5 promoters (FIG. 3b, c).

Androgen-induced phosphorylation at H3T6 is PKCβI-dependent since it is blocked by knockdown of PKCβI. PKCβI depletion is specific and does not affect the levels of endogenous AR (FIG. 3c, right panel). PKCβI depletion does neither affect ligand-dependent recruitment of AR nor the presence of LSD1 on AR-regulated genes (FIG. 3b, c). Since PKCβI is present on the promoter of AR target genes irrespective of the presence or absence of ligand, whereas H3T6 phosphorylation is only observed in the presence of R1881, it was investigated whether PKCβI is phosphorylated and thereby activated in an androgen-dependent manner. Active PKCβI is characterised by phosphorylated serine 660¹² (S660ph). ChIP assays using α-PKCβI S660ph antibody show androgen-induced phosphorylation at 5660 of PKCβI at the KLK2 and FKBP5 promoters (FIG. 3b, c). Taken together, these data demonstrate androgen-dependent activation of PKCβI and phosphorylation of H3T6 by PKCβI in vivo.

### Example 5

### PKCβI Induced Phosphorylation at H3T6 is Controlled by PRK1

It was recently shown by the inventors that PRK1 is a gatekeeper for regulation of AR target gene expression³. PRK1 controls multiple processes during AR-dependent transcription, such as transition from the RNA polymerase II pre-initiation to the initiation complex and demethylation at lysine 9 of histone H3 (H3K9)³. Therefore, it was tested whether in the presence of ligand PKCβI and PRK1 are present in the same complex at promoters of AR target genes. Re-ChIP analysis first with α-PKCβI and then with α-PRK1 antibody demonstrates that both proteins form a complex on the KLK2 and FKBP5 promoters upon addition of ligand (FIG. 3a, lower panel). It was next investigated whether PRK1 regulates PKCβI activity. LNCaP cells, grown in the presence or absence of R1881, were transfected with either an unrelated control siRNA or a siRNA directed against PRK1 and then subjected to ChIP. Ligand-dependent activation of PKCβI is blocked by knockdown of PRK1 (FIG. 3d, e). Consequently, ligand-dependent phosphorylation of H3T6 at the AREs of the KLK2 and FKBP5 promoters is abolished (FIG. 3d, e). PRK1 depletion is specific and does neither affect the levels of endogenous AR (FIG. 3e, right panel) nor the presence of PKCβI on AR target promoters (FIG. 3d, e). In summary, these data demonstrate that PKCβIphosphorylates H3T6 on chromatinized promoters of AR target genes in an androgen- and PRK1-dependent manner.

### Example 6

### PKCβI Induced Phosphorylation at H3T6 Blocks Demethylation of Methylated H3K4me2 and H3K4me1 During AR Induced Gene Expression

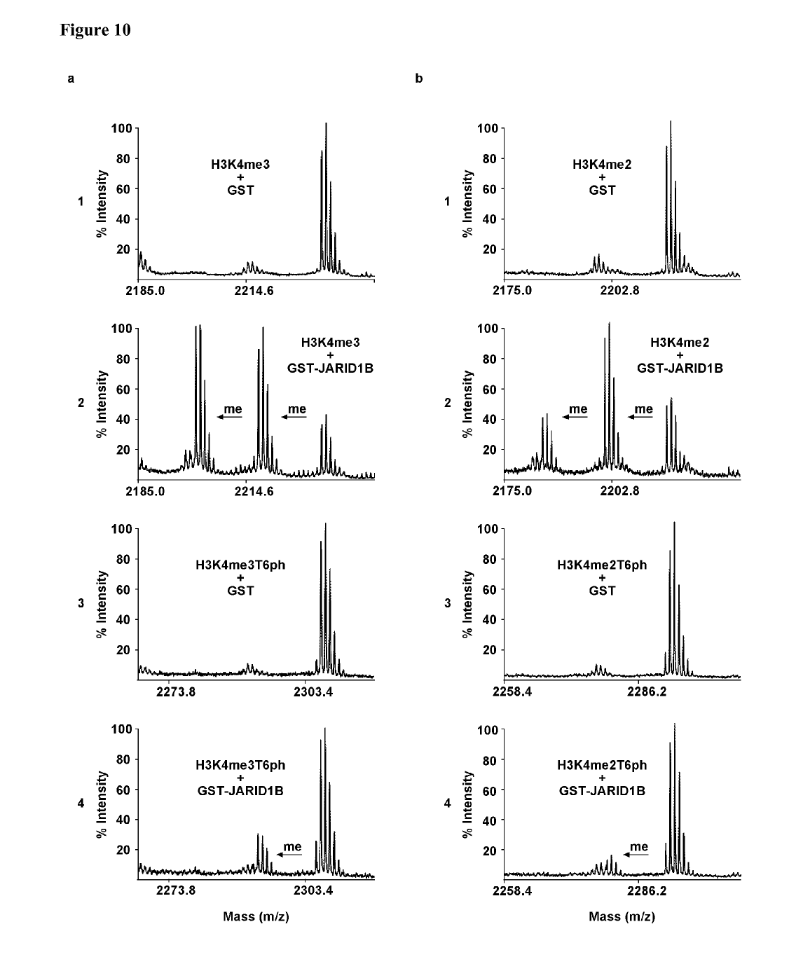

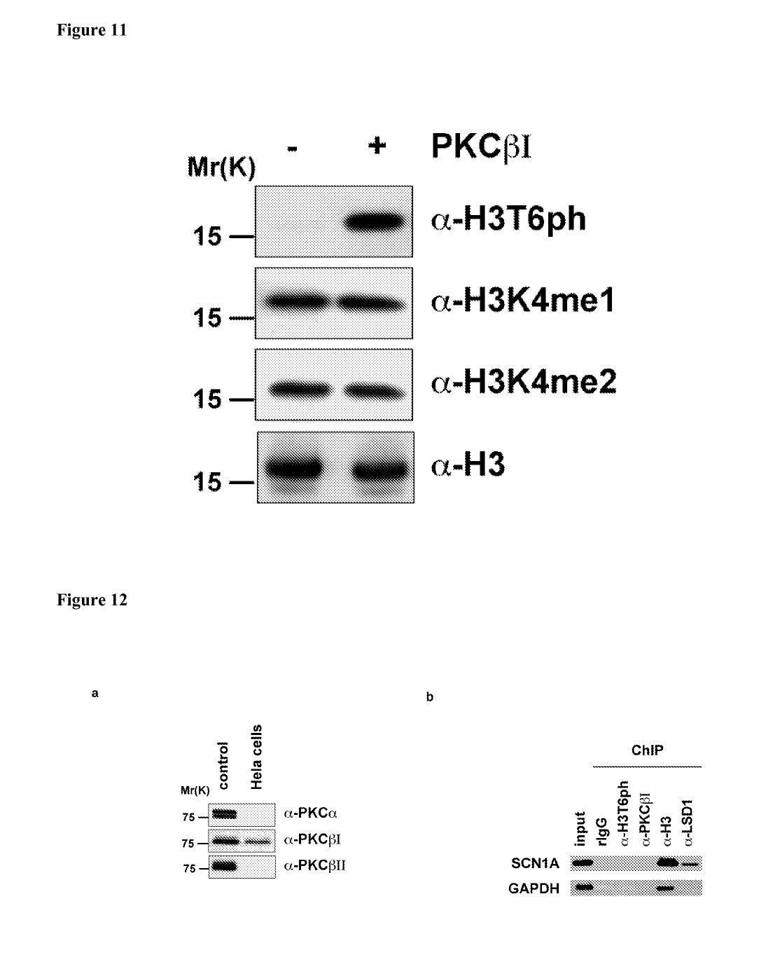

It was analysed whether PKCβI protects demethylation of H3K4 at androgen regulated promoters in vivo. LNCaP cells were cultivated in the presence or absence of R1881, transfected with either an unrelated control siRNA or a siRNA directed against PKCβI and subjected to ChIP. As shown in FIGS. 3f and 3g, levels of H3K4me2 and H3K4me1 observed at the KLK2 and FKBP5 promoters in the absence of ligand are increased upon the addition of R1881, which is in agreement with previous data¹³. Furthermore, the ligand-dependent increase in H3K4me2 and H3K4me1 correlates with R1881-induced phosphorylation of H3T6 by PKCβI. Knockdown of PKCβI blunts ligand-induced phosphorylation of H3T6 and reduces H3K4me2 and H3K4me1 levels to those observed in the absence of R1881. Antibody recognition of H3K4 methyl marks is not altered by phosphorylation at H3T6 (FIG. 11). Together, said observations show that phosphorylation of H3T6 by PKCβI interferes with the removal of H3K4 methyl marks by LSD1 in vivo. Elevated levels of H3K4me2 and H3K4me1 are observed upon PKCβI knockdown in the absence of R1881 indicating androgen-independent functions of PKCβI beyond phosphorylation of H3T6 (FIG. 3f, g). Next, it was tested whether PKCβI signalling also occurs on genes such as SCN1A or SCN2A where LSD1 removes active H3K4 methyl marks to silence gene expression'. ChIP assays performed in LNCaP and HeLa cells show that opposite to AR-activated genes, PKCβI does not associate with LSD1-silenced genes and consequently H3T6 is not phosphorylated (FIG. 3h, i and FIG. 12a, b).

### Example 7

### PKCβI Plays an Important Role in the Control of AR-Dependent Tumour Cell Growth

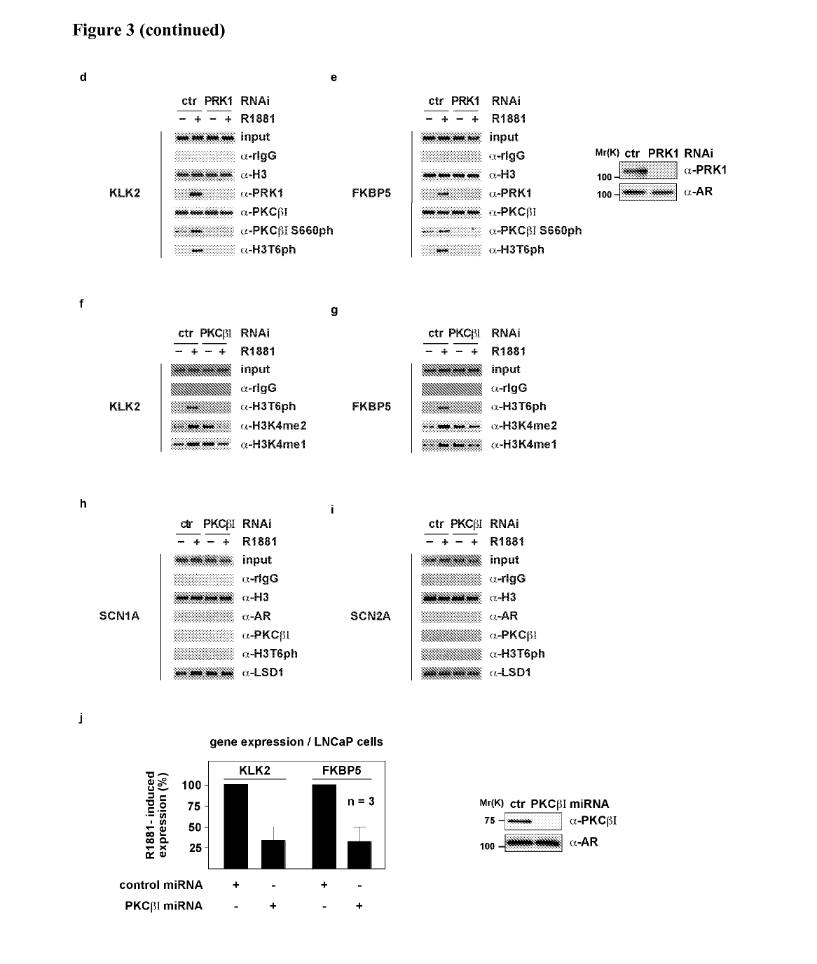

To assess the physiological role of PKCβI, the levels of PKCβI, H3T6ph and H3K4me2 were analysed by immunostaining a panel of 25 normal human prostates and 154 prostate carcinomas on tissue microarrays (FIG. 4 a-d and f). Quantification of immunoreactivity by scoring staining intensity and percentage of positive carcinoma cells¹⁴ reveals that high PKCβI, H3T6ph, and H3K4me2 levels significantly correlate with high Gleason scores and indicate aggressive biology of the tumours (FIG. 4b-d and f). To examine whether PKCβI regulates tumour cell proliferation, androgen-dependent proliferation of LNCaP cells was quantified upon PKCβI knockdown by RNAi. Androgen-induced proliferation of LNCaP cells is severely reduced by PKCβI knockdown when compared to control cells (FIG. 4e), emphasising the important role of PKCβI in the control of AR-dependent tumor growth.

### LITERATURE

1. Shi, Y. et al. Histone demethylation mediated by the nuclear amine oxidase homolog LSD1. Cell 119, 941-953. (2004).
2. Metzger, E. et al. LSD1 demethylates repressive histone marks to promote androgen-receptor-dependent transcription. Nature 437, 436-439. (2005).
3. Metzger, E. et al. Phosphorylation of histone H3 at threonine 11 establishes a novel chromatin mark for transcriptional regulation. Nat. Cell Biol. 10, 53-60. (2008).
4. Strahl, B. D. & Allis, C. D. The language of covalent histone modifications. Nature 403, 41-45. (2000).
5. Klose, R. J., Kallin, E. M. & Zhang, Y. JmjC-domain-containing proteins and histone demethylation. Nat. Rev. Genet. 7, 715-727. (2006).
6. Wissmann, M. et al. Cooperative demethylation by JMJD2C and LSD1 promotes androgen receptor-dependent gene expression. Nat. Cell Biol. 9, 347-353. (2007).
7. Yang, M. et al. Structural basis of histone demethylation by LSD1 revealed by suicide inactivation. Nat. Struct. Mol. Biol. 14, 535-539. (2007).
8. Formeris, F., Binda. C., Adamo, A., Battaglioli, E. & Mattevi, A. Structural basis of LSD1-CoREST selectivity in histone H3 recognition. J. Biol. Chem. 282, 20070-20074. (2007).
9. Metzger, E. & Schüle R. The expanding world of histone lysine demethylases. Nat. Struct. Mol. Biol. 14, 252-254. (2007).
10. Kang, Z., Pirskanen, A., Janne, O. A. & Palvimo, J. J. Involvement of proteasome in the dynamic assembly of the androgen receptor transcription complex. J. Biol. Chem. 277, 48366-48371. (2002).
11. Magee, J. A. et al. Direct, androgen receptor mediated regulation of the FKBP5 gene via a distal enhancer element. Endocrinology 147, 590-598. (2005).
12. Keranen L. M., Dutil E. M. & Newton A. C. Protein kinase C is regulated in vivo by three functionally distinct phosphorylations. Curr. Biol. 5, 1394-1403. (1995).
13. Kang, Z., Jänne O. A. & Palvimo J. J. Coregulator recruitment and histone modifications in transcriptional regulation by the androgen receptor. Mol. Endocrinol. 18, 2633-2648. (2004).
14. Kononen J. et al. Tissue microarrays for high-throughput molecular profiling of tumour specimens. Nat. Med. 4, 844-847. (1998).
15. Wiznerowicz, M. & Trono, D. Conditional suppression of cellular genes: lentivirus vector-mediated drug-inducible RNA interference. J. Virol. 77, 8957-8961. (2003).
16. O'Neill, T. E., Roberge, M. & Bradbury, E. M. Nucleosome arrays inhibit both initiation and elongation of transcripts by bacteriophage T7 RNA polymerase. J. Mol. Biol. 223, 67-78. (1992).
17. Kahl, P. et al. Androgen receptor coactivators lysine-specific histone demethylase 1 and four and a half LIM domain protein 2 predict risk of prostate cancer recurrence. Cancer Res. 66, 11341-11347. (2006).
18. Schroder, F. H. et al. The TNM classification of prostate cancer. Prostate Suppl. 4, 129-138. (1992).
19. Hsing, A. W., Tsao L. & Devesa S. S. International trends and patterns of prostate cancer incidence and mortality. Int. J. Cancer 85, 60-67. (2000).
20. Tsukada, Y. I. et al. Histone demethylation by a family of JmjC domain-containing proteins. Nature 435, 811-816. (2005).
21. Dai, J. et al. The kinase haspin is required for mitotic histone H3 Thr 3 phosphorylation and normal metaphase chromosome alignment. Genes Dev. 19, 472-488. (2005).

1-15. (canceled)

16. A method for detecting, grading and/or prognosticating cancer comprising the step of determining in a sample from a subject the amount of histone H3 phosphorylated at threonine 6 (H3T6ph), the amount of histone H3 monomethylated at lysine 4 (H3K4me1), the amount of histone H3 dimethylated at lysine-4 (H3K4me2) and/or the amount of histone H3 trimethylated at lysine 4 (H3K4me3).

17. A method according to claim 16, further comprising the step of comparing the amount of histone H3 phosphorylated at threonine 6 (H3T6ph), the amount of histone H3 monomethylated at lysine 4 (H3K4me1), the amount of histone H3 dimethylated at lysine 4 (H3K4me2) and/or the amount of histone H3 trimethylated at lysine 4 (H3K4me3) determined in the sample from the subject to the amount of the respective compound(s) determined in a control.

18. A method for detecting, grading and/or prognosticating cancer comprising the step of determining in a sample from a subject the expression level of protein kinase C beta 1 and/or 2 (PKCβI and/or II).

19. A method according to claim 18, further comprising the step of comparing the expression level of protein kinase C beta 1 and/or 2 (PKCβI and/or II) determined in the sample from the subject to the expression level of protein kinase C beta 1 and/or 2 (PKCβI and/or II) in a control.

20. The method according to claim 16 or 18, wherein the sample from the subject is a body fluid or tissue sample, preferably a prostate or breast tissue sample.

21. The method according to claim 20, wherein the body fluid is selected from the group of blood, plasma, urine, saliva, serum, semen, prostate fluid or seminal fluid.

22. A diagnostic kit for detecting, grading and/or prognosticating cancer comprising a detecting agent specific for protein kinase C beta 1 and/or 2 (PKCβI and/or II), histone H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3).

23. An isolated polynucleotide comprising or consisting of SEQ ID NO: 1 or 2 or a fragment or derivative thereof, wherein said polynucleotide is suitable for inhibiting the expression or activity of protein kinase C beta 1 (PKCβI) and/or protein kinase C beta 2 (PKCβII).

24. An isolated polynucleotide according to claim 23, wherein said isolated polynucleotide is a small interfering RNA (siRNA) or a micro RNA (miRNA).

25. A pharmaceutical composition comprising at least one compound selected from
(a) an siRNA molecule suitable for reducing or inhibiting the expression of protein kinase C beta 1 (PKCβI) and/or protein kinase C beta 2 (PKCβII); and/or
(b) the isolated polynucleotide according to any of claim 23 or 24.

26. A method for treating or preventing cancer comprising administering to a subject in need of treatment or prevention an effective amount of the pharmaceutical composition according to claim 25.

27. Use of a detecting agent specific for protein kinase C beta 1 and/or 2 (PKCβI and/or II), historic H3 phosphorylated at threonine 6 (H3T6ph), histone H3 monomethylated at lysine 4 (H3K4me1), histone H3 dimethylated at lysine 4 (H3K4me2) or histone H3 trimethylated at lysine 4 (H3K4me3) for detecting, grading and/or prognosticating cancer in a sample from a subject.

28. The diagnostic kit according to claim 22, wherein the detecting agent is an antibody, an aptamer or an oligonucleotide probe.

29. The use according to claim 27, wherein the detecting agent is an antibody, an aptamer or an oligonucleotide probe.

30. The method according to claim 16 or 18, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.

31. The diagnostic kit according to claim 22, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.

32. The method according to claim 26, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.

33. The use according to claim 27, wherein the cancer is selected from the group consisting of prostate cancer, breast cancer, lung cancer, pancreatic cancer, neuroblastoma, melanoma, brain cancer, kidney cancer, bladder cancer, ovarian cancer, blood cancer and colon cancer.
